(19) United States
(12) Patent Application Publication
(10) Pub. No.: US 2021/0380552 A1
(43) Pub. Date: Dec. 9, 2021

(54) APPARATUS FOR CANNABINOL GENERATION AND METHODS OF USING THE SAME

(71) Applicant: AZDT Holdings, LLC, North Miami Beach, FL (US)

(72) Inventor: Tony M. VERZURA, Auburndale, MA (US)

(21) Appl. No.: 17/411,607

(22) Filed: Aug. 25, 2021

Related U.S. Application Data
(63) Continuation of application No. PCT/US2021/032586, filed on May 14, 2021.
(60) Provisional application No. 63/024,740, filed on May 14, 2020.

(51) Int. Cl. C07D 311/80

(52) U.S. Cl. CPC C07D 311/80

(57) ABSTRACT

The present disclosure methods for producing cannabinol from Cannabis compositions. The present disclosure further provides apparatuses for the production of cannabinol from Cannabis compositions.

### CROSS-REFERENCE To RELATED APPLICATIONS

This application is a continuation of International Application No. PCT/US2021/032586, filed on May 14, 2021. International Application No. PCT/US2021/032586 claims the benefit of priority to U.S. Provisional Application No. 63/024,740, filed on May 14, 2020. Each of the aforementioned applications is hereby incorporated by reference in its entirety for all purposes.

### FIELD

The present disclosure generally describes methods of modulating cannabinoid content within Cannabis compositions. Methods of converting Δ-9-tetrahydrocannabinol (THC) to cannabinol and apparatuses used for said methods are described herein.

### BACKGROUND

Cannabis provides substantial and varied medical benefits. Cannabis contains over 400 molecules, including cannabinoids and terpenes, which may impart the aforementioned medical benefits. One such cannabinoid, cannabinol (CBN), is a potential promising new therapeutic. CBN has been shown to alleviate insomnia, inhibit proliferation of cancer cells, and kill antibiotic resistant bacteria, including methicillin-resistant Staphylococcus aureus.

However, in contrast to other cannabinoids, such as cannabidiol (CBD) and tetrahydrocannabinol (THC), limited studies have explored CBN's potential to treat disease. Obtaining CBN is challenging, because CBN is present at trace levels in Cannabis. Current methods for producing CBN utilize harsh reagents, including sulfur, chloranyl (tetrachloro-1,4-benzoquinone), and iodine. These methods introduce impurities into CBN compositions which are difficult to remove and toxic. Thus, there is an unmet need in the art for improved methods of producing CBN. Such methods will facilitate research and enjoyment of CBN's therapeutic properties.

### SUMMARY

In some embodiments, the present disclosure provides methods of producing cannabinol without the use of physical catalysts. The present disclosure provides methods and apparatuses for reducing THC content in Cannabis compositions. The disclosure further provides methods and apparatuses for increasing cannabinol content within a Cannabis composition.

In some embodiments, provided herein is a method for converting a Δ⁹-tetrahydrocannabinol (THC) source to cannabinol (CBN), said method comprising the steps of:

(a) providing a THC source, an ultra violet (UV) light source, a heat source, and an oxygen source;

(b) exposing the THC source to UV light and heat in an oxygen-rich environment for a time period sufficient to convert the THC in the THC source to CBN.

In some embodiments, the UV light produces long wave UV.

In some embodiments, the UV light produces light at a wavelength between about 315 nm and about 415 nm.

In some embodiments, the UV light produces light at a wavelength of about 403 nm.

In some embodiments, step (b) of the method provided herein is conducted at a temperature of between about 130° C. and about 180° C. In some embodiments, step (b) of the method provided herein is conducted at a temperature of between about 150° C. and about 170° C. In some embodiments, step (b) of the method provided herein is conducted at a temperature of 160° C.

In some embodiments, the THC source is stirred during step (b) of the method provided herein.

In some embodiments, the time period of step (b) of the method provided herein is at least about 10, 9, 8, 7, 6, 5, 4, 3, 2, or 1 days.

In some embodiments, the time period of step (b) of the method provided herein is at least about 24, 48, or 72 hours.

In some embodiments, the THC source comprises tetrahydrocannabinolic acid (THCA), which is then converted to THC in step (b) of the method provided herein.

In some embodiments, the THC source comprises cannabidiol (CBD) and/or cannabidiolic acid (CBDA), which is then converted to THC in step (b).

In some embodiments, the THC source comprises THC.

In some embodiments, provided herein is an apparatus for converting a Δ⁹-tetrahydrocannabinol (THC) source to cannabinol (CBN), said apparatus comprising:

(a) an ultra violet (UV) light source;

(b) an oxygen source with an opening through which oxygen is released;

(c) a container with an inside capable of holding liquid; and

(d) a heat source;

wherein the UV source is located so as to shine UV light inside the container; wherein the oxygen source provides oxygen to the inside of the container; and wherein the heat source is located so as to be able to heat the liquid inside the container.

In some embodiments, an apparatus provided herein comprises a stirring mechanism.

In some embodiments, the stirring mechanism is capable of stirring liquids placed inside the container.

In some embodiments, the stirring mechanism is a magnetic mechanism capable of rotating a magnetic stir bar.

In some embodiments, the apparatus comprises a container containing an opening that permits CO₂ to escape the liquid.

In some embodiments, the UV light source of the apparatus provided herein produces long wave UV light.

In some embodiments, the UV light source of the apparatus provided herein produces light at a wavelength between about 315 nm and about 415 nm.

In some embodiments, the UV light source of the apparatus provided herein produces light at a wavelength of about 403 nm.

In some embodiments, the apparatus comprises a temperature monitor located so as to measure the temperature of the liquid inside the container.

In some embodiments, the temperature monitor is operably connected to the heat source.

In some embodiments, the temperature monitor is configured to regulate the heat source so as to maintain the liquid inside of the container at a temperature between 130° C. and 180° C.

In some embodiments, the oxygen source comprises an aeration stone.

In some embodiments, the aeration stone is located on the opening through which oxygen is released.

In some embodiments, the aeration stone is a 0.5 μm stone.

In some embodiments, the container comprises a cannabinoid selected from the group consisting of THC, tetrahydrocannabinolic acid (THCA), cannabidiol (CBD), cannabidiolic acid (CBDA), and CBN.

Provided herein is a method for converting a Δ⁹-tetrahydrocannabinol (THC) source to cannabinol (CBN), said method comprising the steps of: a) providing a THC source, an ultra violet (UV) light source, and a heat source; b) exposing the THC source to UV light from the UV light source and heating the THC source using the heat source for a time period sufficient to convert THC in the THC source to CBN. In some embodiments, an oxygen flow source is provided in step (a), and wherein step (b) comprises exposing the THC source to exogenous oxygen from the oxygen source. In some embodiments, the THC source is mixed/agitated/stirred during step (b). In some embodiments, the UV light comprises long-wave UV light. In some embodiments, the UV light comprises UV-A light. In some embodiments, the UV light comprises light at a wavelength between about 315 nm and about 415 nm. In some embodiments, the UV light comprises light at a wavelength of about 403 nm. In some embodiments, the UV light has an irradiance in the UV-A range, of greater than 10 W/m². In some embodiments, step (b) is conducted at a temperature greater than 100° C., 110° C., 120° C., or 130° C. In some embodiments, step (b) is conducted at a temperature of between about 130° C. and about 180° C. In some embodiments, step (b) is conducted at a temperature of between about 150° C. and about 170° C. In some embodiments, step (b) is conducted at a temperature of about 160° C. In some embodiments, the THC source is stirred during step (b). In some embodiments, the time period is at least about 1, 2, 3, 4, 5, 6, 7, 8, 9, or 10 hours. In some embodiments, the time period is at least about 24, 48, or 72 hours. In some embodiments, the THC source comprises tetrahydrocannabinolic acid (THCA), which is then converted to THC in step (b). In some embodiments, the THC source comprises cannabidiol (CBD) and/or cannabidiolic acid (CBDA). In some embodiments, the method comprises isolating the CBN produced from the method. In some embodiments, the method comprises isolating the CBN produced from the method, wherein the isolating step comprises applying chemical separation techniques to recover CBN from the THC source after the time period sufficient to produce CBN. In some embodiments, step (b) produces a CBN percent yield of at least 30%, 40%, or 50%. In some embodiments, step (b) produces a CBN cannabinoid titer of at least 30%, 40%, or 50%. In some embodiments, conversion of THC to CBN in step(b) exhibits a rate constant of at least about 0.06 hr⁻¹. In some embodiments, exogenous oxygen from the oxygen source is delivered inside the THC source, so as to produce exogenous oxygen bubbles. In some embodiments, exogenous oxygen comprises air with an oxygen content of at least 21% by weight. In some embodiments, the exogenous oxygen comprises air with an enriched oxygen content that is greater than the oxygen content of atmospheric air.

Provided herein is a method for converting a Δ⁹-tetrahydrocannabinol (THC) source to cannabinol (CBN), said method comprising the steps of: a) providing a THC source, an ultra violet (UV) light source, a heat source, and an oxygen source; b) exposing the THC source to UV light from the UV light source, exogenous oxygen from the oxygen source, and heat from the heat source for a time period sufficient to convert THC in the THC source to CBN. In some embodiments, the UV light comprises long-wave UV light. In some embodiments, the UV light comprises UV-A light. In some embodiments, the UV light comprises light at a wavelength between about 315 nm and about 415 nm. In some embodiments, the UV light comprises light at a wavelength of about 403 nm. In some embodiments, the UV light has an irradiance in the UV-A range, of greater than 10 W/m². In some embodiments, step (b) is conducted at a temperature greater than 100° C., 110° C., 120° C., or 130° C. In some embodiments, step (b) is conducted at a temperature of between about 130° C. and about 180° C. In some embodiments, step (b) is conducted at a temperature of between about 150° C. and about 170° C. In some embodiments, step (b) is conducted at a temperature of about 160° C. In some embodiments, the THC source is stirred during step (b). In some embodiments, the time period is at least about 1, 2, 3, 4, 5, 6, 7, 8, 9, or 10 hours. In some embodiments, the time period is at least about 24, 48, or 72 hours. In some embodiments, the THC source comprises tetrahydrocannabinolic acid (THCA), which is then converted to THC in step (b). In some embodiments, the THC source comprises cannabidiol (CBD) and/or cannabidiolic acid (CBDA).

In some embodiments, the method comprises isolating the CBN produced from the method. In some embodiments, the method comprises isolating the CBN produced from the method, wherein the isolating step comprises applying chemical separation techniques to recover CBN from the THC source after the time period sufficient to produce CBN. In some embodiments, step (b) produces a CBN percent yield of at least 30%, 40%, or 50%. In some embodiments, step (b) produces a CBN cannabinoid titer of at least 30%, 40%, or 50%. In some embodiments, conversion of THC to CBN in step(b) exhibits a rate constant of at least about 0.06 hr⁻¹. In some embodiments, exogenous oxygen from the oxygen source is delivered inside the THC source, so as to produce exogenous oxygen bubbles. In some embodiments, exogenous oxygen comprises air with an oxygen content of at least 21% by weight. In some embodiments, the exogenous oxygen comprises air with an enriched oxygen content that is greater than the oxygen content of atmospheric air.

In some embodiments, provided herein is a method for converting a Δ⁹-tetrahydrocannabinol (THC) source to cannabinol (CBN), said method comprising the steps of: a) heating a THC source, to at least 100° C., 110° C., 120° C., or 130° C.; and b) shining ultraviolet (UV) light on the THC source; wherein step (a) and (b) are conducted for a time period sufficient to convert THC in the THC source to CBN. In some embodiments, step (c) comprises contacting the THC source with exogenous oxygen, wherein steps (a)-(c) are conducted for a time period sufficient to convert THC in the THC source to CBN. In some embodiments, the exogenous oxygen is delivered inside the THC source, so as to produce exogenous oxygen bubbles within the THC source. In some embodiments, the THC source is mixed/agitated/stirred during the time period sufficient to convert THC in the THC source to CBN. In some embodiments, the UV light comprises long-wave UV light. In some embodiments, the UV light comprises UV-A light. In some embodiments, the UV light comprises light at a wavelength between about 315 nm and about 415 nm. In some embodiments, the UV light comprises light at a wavelength of about 403 nm. In some embodiments, the UV light has an irradiance in the UV-A range, of greater than 10 W/m². In some embodiments, step (b) comprises heating the THC source to a temperature between about 130° C. and about 180° C. In some embodiments, step (b) comprises heating the THC source to a temperature between about 150° C. and about 170° C. In some embodiments, step (b) comprises heating the THC source to a temperature of about 160° C. In some embodiments, the THC source is stirred during the time period sufficient to convert THC in the THC source to CBN. In some embodiments, the time period is at least about 1, 2, 3, 4, 5, 6, 7, 8, 9, or 10 hours. In some embodiments, the time period is at least about 24, 48, or 72 hours. In some embodiments, the THC source comprises tetrahydrocannabinolic acid (THCA), which is then converted to THC in step (b). In some embodiments, the THC source comprises cannabidiol (CBD) and/or cannabidiolic acid (CBDA). In some embodiments, the method produces a CBN percent yield of at least 30%, 40%, or 50%. In some embodiments, the method produces a CBN cannabinoid titer of at least 30%, 40%, or 50%. In some embodiments, conversion of THC to CBN in step (b) exhibits a rate constant of at least about 0.06 hr⁻¹.

In some embodiments, provided herein is an apparatus for converting a Δ⁹-tetrahydrocannabinol (THC) source to cannabinol (CBN), said apparatus comprising: a) a container with an inside compartment capable of holding liquid; b) an ultra violet (UV) light source located so as to shine UV light to the inside compartment of the container; c) an exogenous oxygen source with an opening through which oxygen is released into the container; and d) a heat source located so as to be able to heat the container. In some embodiments, the apparatus comprises a stirring mechanism. In some embodiments, the stirring mechanism is capable of stirring materials placed inside the container. In some embodiments, the stirring mechanism is a magnetic mechanism capable of rotating a magnetic stir bar. In some embodiments, the apparatus comprises a mechanism capable of agitating the container. In some embodiments, the apparatus comprises a pump capable of circulating contents in the container. In some embodiments, the apparatus comprises a motor or servo connected to the container so as to be able to rotate or invert the container. some embodiments, the apparatus comprises a rotary shaker base capable of rotating the container. In some embodiments, the container contains an opening or valve that permits CO₂ to escape the container. In some embodiments, the UV light source produces long wave UV. In some embodiments, the UV light source produces UV-A light. In some embodiments, the UV light source produces light at a wavelength between 315 nm and 415 nm. In some embodiments, the UV light source produces light at a wavelength of 403 nm. In some embodiments, the UV light source produces an irradiance in the UV-A range, of greater than 10 W/m². In some embodiments, the apparatus comprises a temperature monitor located so as to measure the temperature of contents inside the container. In some embodiments, the temperature monitor is operably connected to the heat source. In some embodiments, the temperature monitor is configured to regulate the heat source so as to maintain the contents inside of the container at a temperature between about 130° C. and about 180° C. In some embodiments, the exogenous oxygen source is configured to deliver oxygen inside the container. In some embodiments, the exogenous oxygen source is configured to deliver oxygen to contents inside the container, thereby generating exogenous oxygen bubbles in the contents. In some embodiments, the exogenous oxygen source comprises an aeration stone. In some embodiments, the aeration stone is located on the opening through which oxygen is released. In some embodiments, the aeration stone is a 0.5 μm stone. In some embodiments, the container comprises a cannabinoid selected from the group consisting of Δ⁹-tetrahydrocannabinol (THC), tetrahydrocannabinolic acid (THCA), cannabidiol (CBD), cannabidiolic acid (CBDA), and cannabinol (CBN). In some embodiments, the container comprises THC source.

### BRIEF DESCRIPTION OF THE DRAWINGS

FIG. 1 shows an exemplary apparatus for converting a THC source to CBN. The apparatus contains an ultraviolet (UV) light source (0100), an oxygen source with an opening through which oxygen is released (0101), a container with an inside capable of holding liquid (0102), a heat source (0103), a stirring mechanism (0104), an opening for CO2 release (0105), and a temperature monitor (0106).

FIG. 2 is a graph showing conversion of a THC source containing CBG, CBD, CBN, and THC to CBN using the apparatus of FIG. 1.

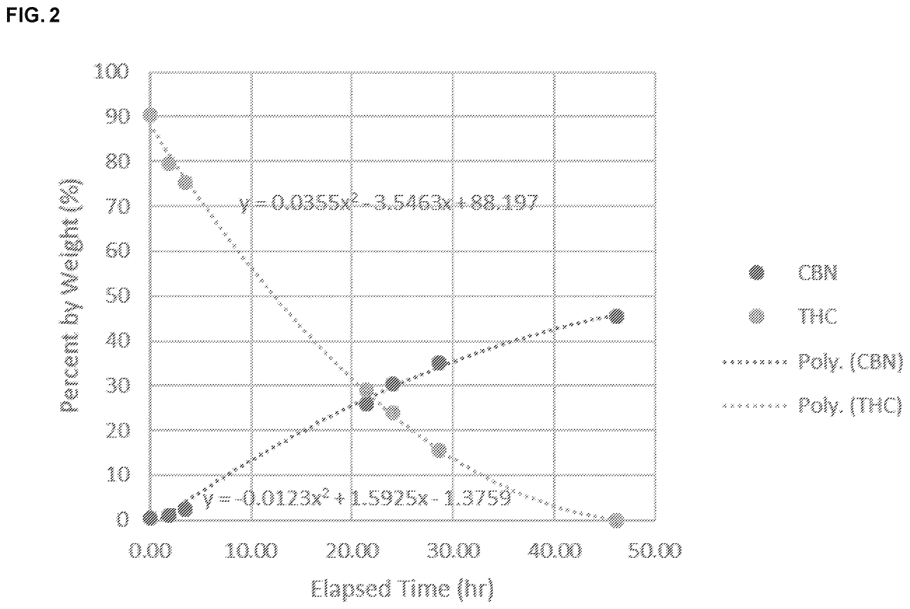

### DETAILED DESCRIPTION

### I. Definitions

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% (i.e., within 10%, 9%, 8%, 7%, 6%, 5%, 4%, 3%, 2%, 1%, or less) above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Herein, the terms “comprise”, or variations such as “comprises” or “comprising”, will be understood to imply the inclusion of a stated element or integer or group of elements or integers but not the exclusion of any other element or integer or group of elements or integers.

The terms “administer,” “administering” or “administration” as used herein refer to either directly administering a compound or pharmaceutically acceptable salt or ester of the compound or a composition comprising the compound or pharmaceutically acceptable salt or ester of the compound to a subject.

The term “Cannabis composition” as used herein refers to any composition containing a cannabinoid. For example, a Cannabis composition may comprise cannabinoids and optionally terpenes. Such cannabinoids and terpenes may be extracted from a Cannabis plant or produced via other chemical or biosynthetic means.

The term “Cannabis plant(s)” encompasses wild type Cannabis sativa and also variants thereof, including Cannabis chemovars which naturally contain different amounts of the individual cannabinoids, Cannabis sativa subspecies indica including the variants var. indica and var. kafiristanica, Cannabis indica and also plants which are the result of genetic crosses, self-crosses or hybrids thereof. The term “Cannabis plant material” is to be interpreted accordingly as encompassing plant material derived from one or more Cannabis plants. For the avoidance of doubt it is hereby stated that “Cannabis plant material” includes dried Cannabis biomass.

As used herein, the term “cultivar” means a group of similar plants that by structural features and performance (i.e., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain and race are often used interchangeably by plant breeders, agronomists and farmers.

As used herein, the term “chemovar” means plants distinguished by the chemical compounds produced, rather than the morphological characteristics of the plant.

The term “enrich” means increasing the amount of a desired ingredient in a composition. For example, enriched oxygen source refers to a gas that contains higher oxygen levels than unaltered atmospheric oxygen. Enriched CBN composition generated by the methods of the present disclosure, refers to a composition comprising higher levels of CBN than it had before carrying out the method.

“Plant material” is defined as a plant or plant part (e.g. bark, wood, leaves, stems, roots, flowers, fruits, seeds, berries or parts thereof) as well as exudates.

As used within the context of this application, the term “purified” means extracted, isolated, and/or separated from other compounds, formulations, compositions, matter, and/or mass. A “purified” substance as described herein is at least about 80%, at least about 85%, at least about 90%, at least about 95%, at least about 96%, at least about 97%, at least about 98%, or at least about 99% pure.

As used herein, the term “winterizing” or “winterization” refers to the process by which plant lipids and waxes are removed from a Cannabis extract. Persons have skill in the art will immediately recognize how to winterize an extract. Briefly, winterization is the dissolving the Cannabis extract into a polar solvent (most commonly ethanol) at cold (e.g., sub-zero Celsius) temperatures. Doing so separates the waxes and lipids from the oil, forcing them to collect at the top of the mixture for easy filtration/collection. Typically, winterization is conducted by mixing ethanol and hash oil into a container and placing it into a sub-zero freezer.

As used herein, the terms, “THC source” or “source of THC” refer to a composition comprising one or more cannabinoids, wherein said cannabinoids either include THC, or can be converted to THC in the course of the methods of the present disclosure. In some embodiments, THC is a THC source. In some embodiments, THCA is a THC source, because THCA is decarboxylated to THC during the methods of the present disclosure. In some embodiments, CBD is a THC source, because CBD can be converted to THC via heat. In some embodiments, CBDA is a THC source, because CBDA is converted to CBD, which is then converted to THC during the methods of the present disclosure.

As used herein, the term “oxygen-rich” environment refers to an environment that contains artificially enriched oxygen levels. In some embodiments, the present disclosure teaches creating oxygen rich environments for Cannabis compositions though means of introducing oxygen (either atmospheric air, or further purified oxygen).

As used herein, the term “yield” refers to the amount of a product (e.g., CBN) obtained per unit weight of a reactant (e.g., THC) and may be expressed as g of product per g of reactant (g/g). As used herein, the term “percent yield” refers to the percent conversion of a reactant (e.g., THC) to a product (e.g., CBN). Percent yield may be calculated according to the following formula: (100×actual yield of CBN)/(theoretical yield of CBN). The theoretical yield of CBN may be calculated provided using stoichiometry. For example, if the THC source is THC, and the initial amount of THC is 500 grams (g), the theoretical yield of CBN is 493.6 g. If the actual yield of CBN is 300 g, then the percent yield is 100%×(300 g/493.6 g)=60.8%.

The term “titre” or “titer” is defined as the strength of a solution or the concentration of a substance in solution. For example, the titre of a product of interest (e.g. small molecule, peptide, synthetic compound, fuel, alcohol, etc.) in a reaction is described as g of product of interest in solution per liter of reaction (g/L).

The term “cannabinoid titre” or “cannabinoid titer” is defined as the strength of a selected cannabinoid in relation to other cannabinoids in a solution. For example, the cannabinoid titre of CBN produced according to the methods of the present disclosure is described as a percentage calculated by dividing the grams of CBN in the final reaction by combined weight of all cannabinoids in the reaction in grams. The term “rate constant” also referred to as “k” refers to a coefficient of proportionality relating the rate of a chemical reaction (e.g. THC→CBN) to the concentration of reactant (e.g., THC). The value of the rate constant relates to the speed of a chemical reaction. If a first reaction has a rate of 10 hr⁻¹, and a second reaction has a rate of 100 hr⁻¹, the second reaction is faster than the first reaction. In some embodiments, the rate constant is calculated by plotting ln (concentration of reactant) vs. time and fitting the plot to the equation of a straight line. The slope of the line is −k.

The term “exogenous air” or “exogenous oxygen” in the context of this disclosure refers to oxygen provided to a reaction (e.g., a THC source being converted to CBN according to the methods of the present disclosure) that goes beyond the oxygen exposure of the reaction to the unaltered atmosphere when sitting in a container. Exogenous oxygen can be provided to a reaction by increasing the oxygen content of the local atmosphere of the container comprising the reaction and/or by flowing air (e.g., atmospheric or oxygen enriched) into the reaction, thereby producing air bubbles in the reaction. As used herein, the term exposing to “exogenous air” or “exogenous oxygen” does not include merely shaking or stirring a reaction in an unaltered atmosphere.

### II. Cannabis Compositions

Provided herein are methods of converting cannabinoids in Cannabis compositions to cannabinol and an apparatus for carrying out said methods. Cannabis compositions are extracts of Cannabis or compositions containing one or more compounds from Cannabis. Cannabis is a genus of flowering plants that includes three different species, Cannabis sativa, Cannabis indica and Cannabis ruderalis. There are 483 identifiable chemical constituents known to exist in the Cannabis plant (Rudolf Brenneisen (2007) in Marijuana and the Cannabinoids, ElSohly, ed.; incorporated herein by reference), and at least 85 different cannabinoids have been isolated from the plant (El-Alfy, Abir T, et al. (2010) Pharmacology Biochemistry and Behavior 95 (4): 434-42; incorporated herein by reference). Cannabis plants are categorized based on the overall amount of THC produced, the ratio of THC to CBD, and the various terpenes produced.

### A. Cannabinoids

Cannabis plants produce a unique family of terpeno-phenolic compounds called cannabinoids. Cannabinoids are a class of diverse chemical compounds that activate cannabinoid receptors in the human brain, peripheral nervous system, and immune system (Mackie K. (2008) J. Neuroendocrinol. May 20: 1:10-4). Cannabinoids can be broadly categorized into endocannabinoids, which are endogenously produced compounds in humans and other animals, such as 2-Arachidonoylglycerol, and phytocannabinoids, which are cannabinoid mimetic compounds produced by plants. Additionally, cannabinoids can be synthesized in a laboratory setting, through chemical or biological means, which can be referred to herein as “synthetic cannabinoids” for greater precision.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms and all cannabinoids in their acid forms (those ending in “A”) can be converted to their non-acidic forms through a process called decarboxylation. While some decarboxylation (e.g., neutralization) of cannabinoids does occur in the plant, production of the neutral forms increase significantly post-harvest with increases in temperature. (Sanchez and Verpoorte (2008) Plant Cell Physiol. Dec: 49(12)). Therefore, decarboxylation is usually achieved by (optionally) thorough drying of the plant material followed by heating it, often by combustion, vaporization, heating, or baking in an oven. Cannabinoid compositions can similarly be decarboxylated by being exposed to heat. Decarboxylation is usually achieved by thorough drying of the plant material followed by heating it, often by either combustion, vaporization, or heating or baking in an oven. In some embodiments, the Cannabis compositions of the present disclosure contain cannabinoids. Unless otherwise noted, references to cannabinoids in this disclosure refer to the “active” decarboxylated versions of the molecules (e.g., CBD or THC).

In order to find the total amount of cannabinoids in a sample (e.g., total amount of active non-acidic cannabinoid), the total measured content of acid cannabinoid variants forms should be adjusted to account for the loss of the carboxyl group. In some embodiments, this adjustment can be made by multiplying the molar content of the acidic cannabinoid forms by the molecular weight of the corresponding decarboxylated cannabinoid. Other shorthand conversions are also available for quickly converting acidic cannabinoid content to active cannabinoid content.

For example, in some embodiments, THCA can be converted to active THC using the formula: THCA×0.877=THC. When using this approach, the maximum THC for the sample is: THC max=(THCA×0.877)+THC. This method has been validated according to the principles of the International Conference on Harmonization. Similarly, CBDA can be converted to active CBD and the yield is determined using the yield formula: CBDA×0.877=CBD. Also, the maximum amount of CBD yielded, i.e. max CBD for the sample is: CBD max=(CBDA×0.877)+CBD. Additionally, CBGA can be converted to active CBG by multiplying CBGA by 0.878 (CBG max=(CBGA×0.878)+CBG). THCVA and CBDVA can be converted to THCV and CBDV, respectively by multiplying their acidic contents by 0.8668 (THCV max=(THCVA×0.8668)+THCV; CBDV max=(CBDVA×0.8668)+CBDV). CBGVA can be converted to CBGV by multiplying CBGVA by 0.8676 (CBGV max=(CBGVA×0.8676)+CBGV). In some embodiments, the Cannabis compositions of the present disclosure contain one or more cannabinoids isolated from a Cannabis plant or produced synthetically, wherein the cannabinoid is selected from the group consisting of Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBDVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C₄ (THCA-C₄), Tetrahydrocannbinol C₄(THC-C₄), Tetrahydrocannabivarinic acid (THCVA), Tetrahydrocannabivarin (THCV), Tetrahydrocannabiorcolic acid (THCA-C₄), Tetrahydrocannabiorcol (THC-C₁), Δ⁷-cis-iso-tetrahydrocannabivarin, Δ⁸-tetrahydrocannabinolic acid (Δ8-THCA), Cannabivarinodiolic (CBNDVA), Cannabivarinodiol (CBNDV), Δ⁸-tetrahydrocannabinol (Δ⁸-THC), Δ⁹-tetrahydrocannabinol (Δ⁹-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C₄ (CBN-C₄), Cannabivarin (CBV), Cannabino-C₂ (CBN-C₂), Cannabiorcol (CBN-C₁), Cannabinodiol (CBND), Cannabinodiolic Acid (CBNDA), Cannabinodivarin (CBDV), Cannabitriol (CBT), 10-Ethoxy-9-hydroxy-Δ^{6a}-tetrahydrocannabinol, 8,9-Dihydroxy-Δ^{6a(10a)}-tetrahydrocannabinol (8,9-Di-OH-CBT-C₅), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ^{6a(10a)}-tetrahydrocannabinol (OTHC), Δ⁹-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide. See Holley et al. (1975) J. Pharm. Sci. 64:892-894 and De Zeeuw et al. (9172) Science 175:778-779, each of which is herein incorporated by reference in its entirety for all purposes.

Brief descriptions and chemical structures for several cannabinoids are provided below.

### Tetrahydrocannabinol

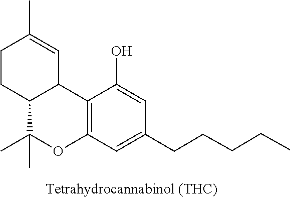

Known as delta-9-tetrahydrocannabinol (Δ9-THC), THC is the principal psychoactive constituent (or cannabinoid) of the Cannabis plant. The initially synthesized and accumulated form in the Cannabis plant is THC acid (THCA). THC has mild to moderate analgesic effects, and Cannabis can be used to treat pain by altering transmitter release on dorsal root ganglion of the spinal cord and in the periaqueductal gray. Other effects include relaxation, alteration of visual, auditory, and olfactory senses, fatigue, and appetite stimulation. THC has marked antiemetic properties, and may also reduce aggression in certain subjects (Hoaken (2003) Addictive Behaviors 28: 1533-1554).

The pharmacological actions of THC result from its partial agonist activity at the cannabinoid receptor CB1, located mainly in the central nervous system, and the CB2 receptor, mainly expressed in cells of the immune system (Pertwee, (2006) International Journal of Obesity 30: S13-S18.) It is also suggested that THC has an anticholinesterase action, which may implicate it as a potential treatment for Alzheimer's and Myasthenia gravis (Eubanks et al., (2006) Molecular Pharmaceutics 3 (6): 773-7).

Non-limiting examples of THC variants include Δ⁹-THC-C₅, Δ⁹-THC-C₄, Δ⁹-THCV-C₃, Δ⁹-THCO-C₁, Δ⁹-THCA-C₅ A, Δ⁹-THCA-C₅ B, Δ⁹-THCA-C₄ A, Δ⁹-THCA-C₄ B, Δ⁹-THCVA-C₃ A, Δ⁹-THCOA-C₁ A, Δ⁹-THCOA-C₁ B, Δ⁸-THC-C₅, Δ⁸-THCA-C₅ A, (−)-cis-Δ⁹-THC-C₅.

In some embodiments, the Cannabis compositions comprise THC or a variant thereof.

### Tetrahydrocannabinolic Acid (THCA)

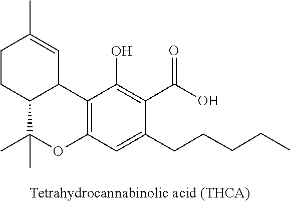

THCA, or 2-COOH-THC, is a major cannabinoid found in Cannabis plant. THCA is produced by cyclization of cannabigerolic acid, a reaction that is catalyzed by the enzyme THC acid synthase. Over time, or when heated, THCA is decarboxylated producing THC. The pathway for THCA biosynthesis is similar to that which produces the bitter acid humulone in hops. See Fellermeier et al., (1998) FEBS Letters 427 (2): 283-5); de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112). In some embodiments, the Cannabis compositions comprise THCA.

### Cannabidiol

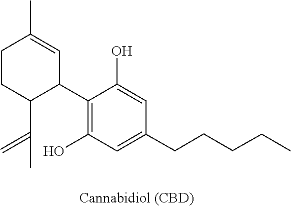 [SMILES: C=C(C)C1CCC(C)=CC1c1c(O)cc(CCCCC)cc1O]

CBD is a cannabinoid found in Cannabis that displays sedative effects in animal tests (Pickens, (1981) Br. J. Pharmacol. 72 (4): 649-56). Some research, however, indicates that CBD can increase alertness, and attenuate the memory-impairing effect of THC. (Nicholson et al., June (2004) J Clin Psychopharmacol 24 (3): 305-13; Morgan et al., (2010) The British Journal of Psychiatry, 197:258-290). It may decrease the rate of THC clearance from the body, perhaps by interfering with the metabolism of THC in the liver. Medically, it has been shown to relieve convulsion, inflammation, anxiety, and nausea, as well as inhibit cancer cell growth (Mechoulam, et al., 2007, Chemistry & Biodiversity 4 (8): 1678-1692), for example reducing growth and invasiveness of aggressive human breast cancer cells (McAllister et al., 2007, Mol. Cancer Ther. 6 (11): 2921-7) Recent studies have also shown CBD to be as effective as atypical antipsychotics in treating schizophrenia (Zuardi et al., 2006, Braz. J. Med. Biol. Res. 39 (4): 421-429.), and studies also suggests that it may relieve symptoms of dystonia (Consroe, 1986, The International journal of neuroscience 30 (4): 277-282).

Cannabis produces CBD-carboxylic acid through the same metabolic pathway as THC, until the last step, where CBDA synthase performs catalysis instead of THCA synthase. See Marks et al. (2009) Journal of Experimental Botany 60 (13): 3715-3726.) and Meijer et al. I, II, III, and IV.

Non-limiting examples of CBD variants include CBD-C₅, CBDM-C₅, CBD-C₄, CBDV-C₃, CBD-C₁, CBDA-C₅, and CBDVA-C₃.

In some embodiments, the Cannabis compositions comprise CBD or a variant thereof.

### Cannabidiolic Acid (CBDA)

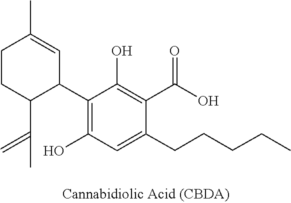 [SMILES: C=C(C)C1CCC(C)=CC1c1c(O)cc(CCCCC)c(C(=O)O)c1O]

CBDA is a major component of Cannabis and is a precursor of cannabidiol. CBDA is converted to CBD through decarboxylation. Studies suggest that CBDA has anti-inflammatory, anti-nausea, and anti-anxiety effects.

In some embodiments, the Cannabis compositions comprise CBDA or a variant thereof.

### Cannabinol

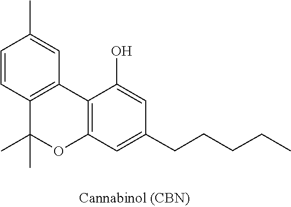 [SMILES: CCCCCc1cc(O)c2c(c1)OC(C)(C)c1ccc(C)cc1-2]

CBN is a mildly to non-psychoactive substance cannabinoid found in Cannabis sativa and Cannabis indica/afghanica. It is also a metabolite of tetrahydrocannabinol (THC). CBN acts as a weak agonist of the CB1 and CB2 receptors, with lower affinity in comparison to THC. Non-limiting examples of CBN variants include CBN-C₅, CBN-C₄, CBN-C₃, CBN-C₂, CBN-C₁, CBNA-C₅ A, and CBNM-C₅.

In some embodiments, the Cannabis compositions comprise CBN. In some embodiments, the methods of the present disclosure enrich Cannabis compositions for CBN. In some embodiments, apparatuses described in the disclosure are used to enrich Cannabis compositions for CBN.

### B. Terpenes and Terpenoids

In addition to cannabinoids, Cannabis produces over 120 different terpenes (Russo (2011) British Journal of Pharmacology, 163:1344-1364). Within the context and verbiage of this document the terms ‘terpenoid’ and ‘terpene’ are used interchangeably. In some embodiments, the present disclosure provides Cannabis compositions comprising one or more terpenes or terpenoids.

In addition to many circulatory and muscular effects, some terpenes interact with neurological receptors. A few terpenes produced by Cannabis plants also bind weakly to cannabinoid receptors. Some terpenes can alter the permeability of cell membranes and allow in either more or less THC, while other terpenes can affect serotonin and dopamine chemistry as neurotransmitters. Terpenoids are lipophilic, and can interact with lipid membranes, ion channels, a variety of different receptors (including both G-protein coupled odorant and neurotransmitter receptors), and enzymes. Some are capable of absorption through human skin and passing the blood brain barrier.

Terpenes are derived biosynthetically from units of isoprene, which have the molecular formula C₅H₈. The basic molecular formulae of terpenes are multiples of (C₅H₈)ₙ where n is the number of linked isoprene units. The isoprene units may be linked together “head to tail” to form linear chains or they may be arranged to form rings. In one embodiment, a Cannabis composition contains a terpene selected from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol. Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, as well as their isomers, enantiomers, or derivatives. Within the context of this disclosure, the term terpene includes the α-(alpha), β-(beta), γ-(gamma), oxo-, isomers, or any combinations thereof.

In some embodiments, a Cannabis composition contains a terpene selected from 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10), 11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In some embodiments, a Cannabis composition of the disclosure contains one or more derivatives of terpenes. Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids. Terpenoids, a.k.a. isoprenoids, are a large and diverse class of naturally occurring organic chemicals similar to terpenes, derived from five-carbon isoprene units assembled and modified in thousands of ways. Non-limiting examples of terpenoids include, Hemiterpenoids, 1 isoprene unit (5 carbons); Monoterpenoids, 2 isoprene units (10 C); Sesquiterpenoids, 3 isoprene units (15 C); Diterpenoids, 4 isoprene units (20 C) (e.g. ginkgolides); Sesterterpenoids, 5 isoprene units (25 C); Triterpenoids, 6 isoprene units (30 C) (e.g. sterols); Tetraterpenoids, 8 isoprene units (40 C) (e.g. carotenoids); and Polyterpenoid with a larger number of isoprene units.

### C. Cannabis Extracts

### Extraction Methods

In some embodiments, the methods and apparatus of the present disclosure utilize a THC source. In some embodiments, the THC source is a Cannabis composition. In some embodiments, a Cannabis composition is extracted from a starting plant material according to methods known in the art. For example, suitable extraction methods include maceration, percolation, solvent extraction, steam distillation (giving you essential oil) or vaporization. General protocols for the preparation of Cannabis compositions from Cannabis plant material are described in U.S. Pat. Nos. 8,603,515 and 9,730,911, both incorporated by reference herein.

Solvent extraction may be carried out using essentially any solvent that dissolves cannabinoids/cannabinoid acids, such as for example C1 to C5 alcohols (e.g. ethanol, methanol), C4-C12 alkanes (e.g. hexane or butane), Norflurane (HFA134a), HFA227, and carbon dioxide. When solvents such as those listed above are used, the resultant primary extract typically contains non-specific lipid-soluble material or “ballast” e.g. waxes, wax esters and glycerides, unsaturated fatty acid residues, terpenes, carotenes, and flavonoids. The primary extract may be further purified for example by “winterization”, which involves chilling to −20° C. followed by filtration to remove waxy ballast, supercritical or subcritical extraction, vaporization, distillation, and chromatography.

In some embodiments, the Cannabis composition may be obtained by carbon dioxide (CO₂) extraction followed by a secondary extraction, e.g. an ethanolic precipitation, to remove a substantial proportion of non-cannabinoid materials. In some embodiments, a Cannabis composition is produced by a process comprising extraction with liquid CO₂ under sub-critical or super-critical conditions, and then a further extraction (e.g., an ethanolic precipitation) to remove significant amounts of ballast. If it is intended to prepare free cannabinoids from the Cannabis plant material, then the material is preferably heated to a defined temperature for a defined period of time in order to decarboxylate cannabinoid acids to free cannabinoids prior to extraction of the botanical drug substance.

In some embodiments, a Cannabis composition is prepared according to a process comprising the following steps: i) optional decarboxylation of the plant material, ii) extraction with liquid CO₂ (in some embodiments under sub-critical conditions), to produce a crude botanical drug substance, iii) precipitation with C1-C5 alcohol to reduce the proportion of non-target materials, iv) removal of the precipitate (preferably by filtration), v) optional treatment with activated charcoal, and vi) evaporation to remove C1-C5 alcohol and water, thereby producing a final botanical drug substance. Extraction techniques for cannabinoids, including vaporizer-based approaches, can be found in U.S. Pat. Nos. 7,700,368, 10,159,908, U.S. Pub. No. 2019/0151771, U.S. Pub. No. 2018/0078874, U.S. Pub. No. 2020/0080021, U.S. Pub. No. 2020/0048214, U.S. Pub. No. 2020/0048215, and U.S. Pat. No. 10,555,914, each of which is incorporated by reference herein in its entirety.

In some embodiments, a Cannabis composition is prepared from plant material according to a process comprising the following steps: i) CO₂ extraction for plant terpenes, ii) ethanol extraction for crude cannabinoids, plant waxes, and plant oils (crude extract); iii) winterization of the crude extract at −80° C. for 24 hours; and iv) complete ethanol recovery and in-vessel decarboxylation of winterized crude before fractional distillation of cannabinoids.

In some embodiments, a Cannabis composition described herein is extracted from Cannabis using any method known in the art and subsequently winterized.

### Selection of Plant Material

Yield of particular cannabinoids or terpenes from extraction varies greatly by plant tissue, type of extraction, age of material, and other variables (McPartland and Russo (2001) “Cannabis and Cannabis Extracts: Greater Than the Sum of Their Parts?” Hayworth Press). The purity of different active ingredients (e.g. cannabinoids and/or terpenes) may be enhanced in extracts by selection of appropriate starting plant material. In some embodiments, plants bred to express desired cannabinoid and/or terpene profiles (i.e., “chemovars”) are selected as the starting plant material for extraction and/or purification. However, it is to be understood that the present disclosure is of general utility and is not limited to the use of particular Cannabis varieties as the starting material.

### Purified Cannabis Compounds/Chemical Separation Techniques for Cannabinoids

In some embodiments, the Cannabis composition extracted from plant material is further purified. In some embodiments, purification comprises various techniques, e.g., chromatography, crystallization, filtration, centrifuge, distillation, or various combinations of said techniques. In one embodiment, “purified” means substantially free from other material, e.g., compounds, particles, vegetative material, plant derived substances, solvents, etc. In one example, the term “purified” refers to a compound purified from a crude extract, such as a biologically derived substance, thereby resulting in a significant difference between the purified compound and the extract.

Within the context of this disclosure, purified compounds may be purposely formulated with other compounds at various levels of purity. For example, depending on the desired outcome, a particular cannabinoid or terpene may be formulated with other molecules when it is 60-65% pure, 65-70% pure, 70-75% pure, 75-80% pure, 80-85% pure, 85-90% pure, 90-95% pure, 95-99% pure, 99-99.9% pure, 99.9+%, or greater than 99% pure.

In some embodiments the purity is determined by area normalization of an HPLC or GC-FID profile.

In some embodiments, a Cannabis composition may contain cannabinoids or terpenes that are produced synthetically.

III. Reactions that Yield CBN

In some embodiments, the disclosure provides methods and apparatuses for producing CBN from a THC source. In some embodiments, the methods and apparatuses described herein convert THC into CBN.

In some embodiments, the THC source is a THC extract or synthetically produced THC. The reaction of THC to CBN is illustrated below (Reaction 1). Aromatization of THC results in the production of CBN. Thus far, aromatization of THC has only been achieved using harsh reagents, such as sulfur, chloranyl (tetrachloro-1,4-benzoquinone), and iodine. Pollastro et al. J. Nat. Prod. 2018, 81, 630-633. These methods are disadvantageous, because they result in the introduction of potentially toxic substances into Cannabis compositions that are utilized recreationally and/or medically. Removal of toxic substances is difficult and costly.

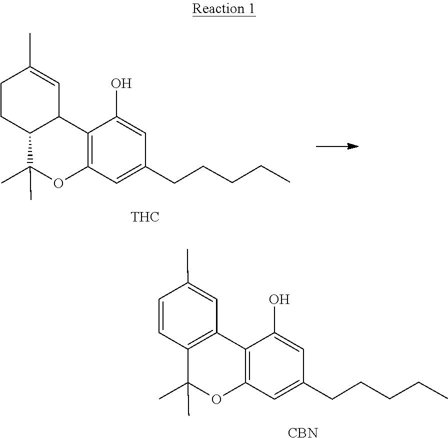

CBD is another THC source. In some embodiments, CBN is produced from CBD. CBN production from CBD requires isomerization of CBD to THC and aromatization of THC to CBN (Reaction 2). Isomerization of CBD to THC has been performed using acids, such as hydrogen chloride, pyridine hydrochloride, phosphoric acid, sulfamic acid, or zinc chloride. Adams et al. J. Am. Chem. Soc. 1940, 62, 9, 2402-2405. Aromatization of THC to CBN has been performed using the techniques described above.

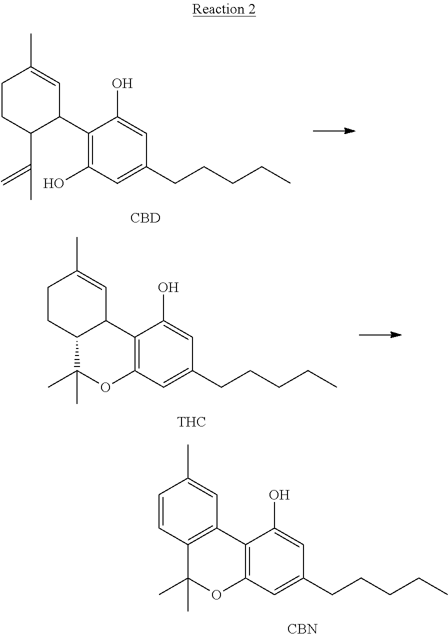

Alternative THC sources comprise CBDA and THCA. In some embodiments, it is envisioned that CBN may be produced from CBDA and THCA. Conversion of precursors CBDA and THCA to CBD and THC requires a decarboxylation step. In some embodiments, decarboxylation of CBDA and/or THCA to CBD and/or THC requires a heating step. In some embodiments, a heating step is performed at a temperature greater than or equal to 100° C., for example, at a temperature of about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., about 121° C., about 122° C., about 123° C., about 124° C., about 125° C., about 126° C., about 127° C., about 128° C., about 129° C., about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., about 148° C., about 149° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., about 180° C., about 181° C., about 182° C., about 183° C., about 184° C., about 185° C., about 186° C., about 187° C., about 188° C., about 189° C., about 190° C., about 191° C., about 192° C., about 193° C., about 194° C., about 195° C., about 196° C., about 197° C., about 198° C., about 199° C., about 200° C., or higher.

### IV. Methods of Producing CBN

In some embodiments, the disclosure provides methods of converting a cannabinoid (e.g. a THC source) to CBN, comprising the steps of: (a) providing said cannabinoid), an ultraviolet (UV) light source, and an oxygen source and (b) exposing the cannabinoid to UV light in an oxygen-enriched environment for a time period sufficient to form the CBN.

In some embodiments, the methods of the disclosure result in the conversion of cannabinoids (e.g. THCA, THC) into CBN without the need for chemical solvents, physical catalysts, solvents, or elemental metals. The use of chemical solvents, physical catalysts, solvents, elemental metals results in a Cannabis composition that has an unpleasant smell and taste and may contain toxins. In some embodiments, the methods of the disclosure are used to create Cannabis compositions with a pleasant taste and/or a pleasant smell.

Furthermore, as described above, in some embodiments, CBN may be produced from a THC source. Exemplary THC sources include THC itself or THCA, CBD, or CBDA, each of which convert to THC through decarboxylation and/or isomerization. Production of CBN from one of the aforementioned cannabinoids results in consumption of THC, THCA, CBD, or CBDA.

Thus, in some embodiments, the methods are utilized to lower the content of THC, THCA, CBD, or CBDA in a Cannabis composition. The methods and apparatus of the present disclosure can be used, in some embodiments, to remediate “hot” Cannabis samples that may contain higher than 0.3% THC by weight. This technique is much cheaper than other THC removal technologies, which require complex separations steps.

In some embodiments, the methods are utilized to provide a defined ratio of a THC source (e.g., THC, THCA, CBD, or CBDA) to CBN in a Cannabis composition. In some embodiments, the methods are utilized to provide a defined ratio of THC to CBN in a Cannabis composition. In some embodiments, the methods are utilized to lower the content of THC to 0.3% or lower by weight of the Cannabis composition.

In some embodiments, a Cannabis composition comprises a THC source that is 95-99% by weight of the Cannabis composition. In some embodiments, a Cannabis composition comprises a THC source that is 85-90% by weight of the Cannabis composition. In some embodiments, a Cannabis composition comprises a THC source that is 75-85% by weight of the Cannabis composition. In some embodiments, a Cannabis composition comprises a THC source that is 55-60% by weight of the Cannabis composition. In some embodiments, the Cannabis composition comprises a THC source that is greater than or equal to 99% by weight of the Cannabis composition. In some embodiments, the Cannabis composition comprises a THC source that is less than or equal to 10% by weight of the Cannabis composition.

In some embodiments, Cannabis compositions (e.g., THC source) contain between about 10% and about 100% THC by weight. For example, the Cannabis compositions contain about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% THC by weight, including all ranges and subranges therebetween. In some embodiments, the Cannabis compositions contain at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, about 96%, at least about 97%, at least about 98%, at least about 99%, or about 100% THC by weight. In some embodiments, the Cannabis compositions contain between about 91% and about 99% of THC by weight. In some embodiments, the Cannabis compositions contain between about 96% and about 99% of THC by weight. In some embodiments, the methods decrease the THC content in a Cannabis composition relative to the THC content of the Cannabis composition prior to performing the method of the present disclosure by about 15% to about 100% by weight. For example, the methods decrease THC content in a Cannabis composition relative to the THC content of the Cannabis composition prior to performing the methods of the present disclosure by about 15%, about 20%, about 25%, about 30%, about 35%, about 40%, about 45%, about 50%, about 55%, about 60%, about 65%, about 70%, about 75%, about 80%, about 85%, about 90%, or 100%, including all subranges and ranges therebetween. In some embodiments, the methods decrease THC content in a Cannabis composition relative to the THC content of the Cannabis composition prior to performing the methods of the present disclosure by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or 100%.

In some embodiments, Cannabis compositions (e.g., THC source) contain between about 10% and about 100% THCA by weight. For example, a Cannabis composition (e.g., THC source) contains about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% THCA by weight, including all ranges and subranges therebetween. In some embodiments, a Cannabis composition contains between about 91% and about 99% of THCA by weight. In some embodiments, a Cannabis composition contains between about 96% and about 99% of THCA by weight. In some embodiments, a Cannabis composition comprising THCA is provided in step (a) and the THCA is converted to THC in step (b). In some embodiments, a Cannabis composition comprising THCA is provided in step (a) and the THCA is converted to THC and then to CBN in step (b).

In some embodiments, the methods decrease the THCA content in a Cannabis composition relative to the THCA content of the Cannabis composition prior to performing the method of the present disclosure by about 15% to about 100% by weight. For example, the methods decrease the THCA content by about 15%, about 20%, about 25%, about 30%, about 35%, about 40%, about 45%, about 50%, about 55%, about 60%, about 65%, about 70%, about 75%, about 80%, about 85%, about 90%, or 100%, including all subranges and ranges therebetween. In some embodiments, the methods decrease THCA content in a Cannabis composition relative to the THCA content of the Cannabis composition prior to performing the methods of the present disclosure by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or 100%.

In some embodiments, a Cannabis composition (e.g., THC source) contains between about 10% and about 100% CBD by weight. For example, a Cannabis composition (e.g., THC source) contains about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% CBD by weight, including all subranges and ranges therebetween. In some embodiments, a Cannabis composition contains between about 91% and about 99% of CBD by weight, including all ranges and subranges therebetween. In some embodiments, a Cannabis composition contains between about 96% and about 99% of CBD by weight. In some embodiments, a Cannabis composition comprising CBD is provided in step (a) and the CBD is converted to THC and then to CBN in step (b).

In some embodiments, the methods decrease the CBD content in a Cannabis composition by about 15% to about 100% by weight relative to the CBD content of the Cannabis composition prior to performing the method of the present disclosure. For example, the CBD content by weight of a Cannabis composition may decrease by about 15%, about 20%, about 25%, about 30%, about 35%, about 40%, about 45%, about 50%, about 55%, about 60%, about 65%, about 70%, about 75%, about 80%, about 85%, about 90%, or 100%, including all ranges and subranges therebetween. In some embodiments, the methods decrease CBD content in a Cannabis composition relative to the CBD content of the Cannabis composition prior to performing the methods of the present disclosure by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or 100%.

In some embodiments, a Cannabis composition (e.g., THC source) contains between about 10% and about 100% CBDA by weight. For example, a Cannabis composition may contain (e.g., THC source) contain about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% CBDA by weight, including all ranges and subranges therebetween. In some embodiments, a Cannabis composition contains between about 91% and about 99% of CBDA by weight. In some embodiments, a Cannabis composition contains between about 96% and about 99% of CBDA by weight. In some embodiments, a Cannabis composition comprising CBDA is provided in step (a) and the CBDA is converted to CBD followed by THC followed by CBN in step (b).

In some embodiments, the methods decrease the CBDA content in a Cannabis composition relative to the CBDA content of the Cannabis composition prior to performing the method of the present disclosure by about 15% to about 100% by weight. For example, the methods decrease the CBDA content in a Cannabis composition relative to the CBDA content of the Cannabis composition prior to performing the method of the present disclosure by about 15%, about 20%, about 25%, about 30%, about 35%, about 40%, about 45%, about 50%, about 55%, about 60%, about 65%, about 70%, about 75%, about 80%, about 85%, about 90%, or 100%, including all subranges and ranges therebetween. In some embodiments, the methods decrease CBDA content in a Cannabis composition relative to the CBDA content of the Cannabis composition prior to performing the methods of the present disclosure by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or 100%.

In some embodiments, the methods convert between about 15% and 100% of a THC source by weight to CBN. For example, the methods convert about 15%, about 20%, about 25%, about 30%, about 35%, about 40%, about 45%, about 50%, about 55%, about 60%, about 65%, about 70%, about 75%, about 80%, about 85%, about 90%, or about 100% of a THC source by weight to CBN, including all ranges and subranges therebetween. In some embodiments, the methods convert at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or 100% of a THC source by weight to CBN.

In some embodiments, the amount of THC source present in a Cannabis composition after performing the methods of the disclosure is less than about 5%, less than about 4%, less than about 3%, less than about 2%, or less than about 1% by weight of the Cannabis composition. In some embodiments, the amount of THC source present in a Cannabis composition after performing the methods of the disclosure is between about 0% and about 1%, between about 1% and about 2%, between about 2% and about 3%, between about 3% and about 4%, between about 4% and about 5%, between about 0% and about 3%, between about 0.1% and about 3%, between about 0% and about 5%, between about 0.1% and about 5%, between about 0% and about 3%, between about 0.1% and about 3%, between about 1% and about 3%, between about 2% and about 4%, between about 3% and about 5%, between about 0% and about 2%, between about 0.1% and about 2%, or between about 4% and about 5% by weight of the Cannabis composition. In some embodiments, the amount of THC source present in a Cannabis composition after performing the methods of the disclosure is between about 0% and about 5% by weight of the Cannabis composition. For example, the amount of a THC source present in a Cannabis composition after performing the methods of the disclosure is 0%, about 0.1%, about 0.2%, about 0.3% , about 0.4%, about 0.5%, about 0.6%, about 0.7%, about 0.8%, about 0.9%, about 1%, about 1.1%, about 1.2%, about 1.3% , about 1.4%, about 1.5%, about 1.6%, about 1.7%, about 1.8%, about 1.9%, about 2%, about 2.1%, about 2.2%, about 2.3% , about 2.4%, about 2.5%, about 2.6%, about 2.7%, about 2.8%, about 2.9%, about 3%, about 3.1%, about 3.2%, about 3.3% , about 3.4%, about 3.5%, about 3.6%, about 3.7%, about 3.8%, about 3.9%, about 4%, about 4.1%, about 4.2%, about 4.3% , about 4.4%, about 4.5%, about 4.6%, about 4.7%, about 4.8%, about 4.9%, or about 5%, including all ranges and subranges therebetween.

In some embodiments, the amount of CBN in a Cannabis composition increases after performing the method of the present disclosure to between about 1% and about 100% by weight of the Cannabis composition. For example, the amount of CBN in a Cannabis composition increases after performing the method of the present disclosure to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% by weight of the Cannabis composition, including all ranges and subranges therebetween. In some embodiments, the amount of CBN in a Cannabis composition increases after performing the method of the present disclosure to at least about 1% by weight of the Cannabis composition. For example, the amount of CBN in a Cannabis composition after performing the methods of the disclosure is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% by weight of the Cannabis composition.

In some embodiments, the amount of CBN in a Cannabis composition increases after performing the method of the present disclosure to between about 15% and about 60%, about 20% to about 55%, about 25% to about 50%, about 30% to about 45%, about 35% to about 40%, about 50% to about 55%, about 40% to about 45%, about 35% to about 45%, or about 20% to about 30% by weight of the Cannabis composition, including all ranges and subranges there between. In embodiments, the amount of CBN in a Cannabis composition increases after performing the method of the present disclosure to between about 15% and about 60% by weight of the Cannabis composition. For example, the amount of CBN in a Cannabis composition increases after performing the method of the present disclosure to about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% by weight of the Cannabis composition including all ranges and subranges therebetween.

In some embodiments, the methods result in an at least 10% to at least about 200% improvement in percent yield of CBN compared to methods that do not use a UV source and/or an oxygen source. For example, the methods result in at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, or more improvement in the percent yield of CBN compared to methods that do not use a UV source and/or an oxygen source, including all ranges and subranges therebetween.

In some embodiments, the methods provide a percent yield of CBN of between about 30% and about 100%. For example, the percent yield of CBN is about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, including all ranges and subranges therebetween. In some embodiments, the percent yield of CBN is at least about 20%, at least about 25%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. In some embodiments, the percent yield is at least about 30%, at least about 40%, or at least about 50%.

In some embodiments, the methods provide a yield of between about 5 milligrams (mg) CBN/g of THC source to about 1000 mg CBN/g of THC source. For example, the methods provide a yield of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg of CBN per g of THC source, including all ranges and subranges therebetween. In some embodiments, the methods provide a yield of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, at least about 200 mg, at least about 205 mg, at least about 210 mg, at least about 215 mg, at least about 220 mg, at least about 225 mg, at least about 230 mg, at least about 235 mg, at least about 240 mg, at least about 245 mg, at least about 250 mg, at least about 255 mg, at least about 260 mg, at least about 265 mg, at least about 270 mg, at least about 275 mg, at least about 280 mg, at least about 285 mg, at least about 290 mg, at least about 295 mg, at least about 300 mg, at least about 305 mg, at least about 310 mg, at least about 315 mg, at least about 320 mg, at least about 325 mg, at least about 330 mg, at least about 335 mg, at least about 340 mg, at least about 345 mg, at least about 350 mg, at least about 355 mg, at least about 360 mg, at least about 365 mg, at least about 370 mg, at least about 375 mg, at least about 380 mg, at least about 385 mg, at least about 390 mg, at least about 395 mg, at least about 400 mg, at least about 405 mg, at least about 410 mg, at least about 415 mg, at least about 420 mg, at least about 425 mg, at least about 430 mg, at least about 435 mg, at least about 440 mg, at least about 445 mg, at least about 450 mg, at least about 455 mg, at least about 460 mg, at least about 465 mg, at least about 470 mg, at least about 475 mg, at least about 480 mg, at least about 485 mg, at least about 490 mg, at least about 495 mg, at least about 500 mg, at least about 505 mg, at least about 510 mg, at least about 515 mg, at least about 520 mg, at least about 525 mg, at least about 530 mg, at least about 535 mg, at least about 540 mg, at least about 545 mg, at least about 550 mg, at least about 555 mg, at least about 560 mg, at least about 565 mg, at least about 570 mg, at least about 575 mg, at least about 580 mg, at least about 585 mg, at least about 590 mg, at least about 595 mg, at least about 600 mg, at least about 605 mg, at least about 610 mg, at least about 615 mg, at least about 620 mg, at least about 625 mg, at least about 630 mg, at least about 635 mg, at least about 640 mg, at least about 645 mg, at least about 650 mg, at least about 655 mg, at least about 660 mg, at least about 665 mg, at least about 670 mg, at least about 675 mg, at least about 680 mg, at least about 685 mg, at least about 690 mg, at least about 695 mg, at least about 700 mg, at least about 705 mg, at least about 710 mg, at least about 715 mg, at least about 720 mg, at least about 725 mg, at least about 730 mg, at least about 735 mg, at least about 740 mg, at least about 745 mg, at least about 750 mg, at least about 755 mg, at least about 760 mg, at least about 765 mg, at least about 770 mg, at least about 775 mg, at least about 780 mg, at least about 785 mg, at least about 790 mg, at least about 795 mg, at least about 800 mg, at least about 805 mg, at least about 810 mg, at least about 815 mg, at least about 820 mg, at least about 825 mg, at least about 830 mg, at least about 835 mg, at least about 840 mg, at least about 845 mg, at least about 850 mg, at least about 855 mg, at least about 860 mg, at least about 865 mg, at least about 870 mg, at least about 875 mg, at least about 880 mg, at least about 885 mg, at least about 890 mg, at least about 895 mg, at least about 900 mg, at least about 905 mg, at least about 910 mg, at least about 915 mg, at least about 920 mg, at least about 925 mg, at least about 930 mg, at least about 935 mg, at least about 940 mg, at least about 945 mg, at least about 950 mg, at least about 955 mg, at least about 960 mg, at least about 965 mg, at least about 970 mg, at least about 975 mg, at least about 980 mg, at least about 985 mg, at least about 990 mg, at least about 995 mg, or at least about 1000 mg of CBN per g of THC source, including all ranges and subranges therebetween.

In some embodiments, the methods produce compositions with CBN titers of between about 10% to about 100%. For example, in some embodiments, the methods produce compositions with CBN titers of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, including all ranges and subranges therebetween. In some embodiments, the methods produce compositions with CBN titers of at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%. In some embodiments, the CBN titer is at least about 30%, at least about 40%, or at least about 50%.In some embodiments, when the methods of the disclosure are performed, the rate constant of conversion of a THC source to CBN is between about 0.01 M/M/hr⁻¹ and about 1 hr⁻¹. For example, the rate constant of conversion of a THC source to CBN is about 0.01 hr⁻¹, about 0.02 hr⁻¹, about 0.03 hr⁻¹, about 0.04 hr⁻¹, about 0.05 hr⁻¹, about 0.06 hr⁻¹, about 0.07 hr⁻¹, about 0.08 hr⁻¹, about 0.09 hr⁻¹, about 0.1 hr⁻¹, about 0.15 hr⁻¹, about 0.2 hr⁻¹, about 0.25 hr⁻¹, 0.30 hr⁻¹, about 0.35 hr⁻¹, about 0.4 hr⁻¹ , about 0.45 hr⁻¹, about 0.5 hr⁻¹, about 0.55 hr⁻¹, about 0.6 hr⁻¹, about 0.65 hr⁻¹, about 0.7 hr⁻¹, about 0.75 hr⁻¹, 0.80 hr⁻¹, about 0.85 hr⁻¹, about 0.9 hr⁻¹, about 0.95 hr⁻¹, or about 1 hr⁻¹, including all subranges and ranges therebetween. In some embodiments, the rate constant of conversion of a THC source to CBN is about 0.06 hr⁻¹.

In some embodiments, the rate constant of conversion of a THC source to CBN is at least about 0.01 hr⁻¹, at least about 0.02 hr⁻¹, at least about 0.03 hr⁻¹, at least about 0.04 hr⁻¹, at least about 0.05 hr⁻¹, at least about 0.06 hr⁻¹, at least about 0.07 hr⁻¹, at least about 0.08 hr⁻¹, at least about 0.09 hr⁻¹, at least about 0.1 hr⁻¹, at least about 0.15 hr⁻¹, at least about 0.2 hr⁻¹, at least about 0.25 hr⁻¹, 0.30 hr⁻¹, at least about 0.35 hr⁻¹, at least about 0.4 hr⁻¹, at least about 0.45 hr⁻¹, at least about 0.5 hr⁻¹, at least about 0.55 hr⁻¹, at least about 0.6 hr⁻¹, at least about 0.65 hr⁻¹, at least about 0.7 hr⁻¹, at least about 0.75 hr⁻¹, 0.80 hr⁻¹, at least about 0.85 hr⁻¹, at least about 0.9 hr⁻¹, at least about 0.95 hr⁻¹, or at least about 1 hr⁻¹, including all subranges and ranges therebetween.

In some embodiments, the rate constant of conversion of a THC source to CBN according to the methods of the disclosure (e.g., when a THC source is exposed to a UV light source and a heat source) is between about 1% and about 500% greater than the rate constant of conversion of a THC source to CBN when either (i) the THC source is only exposed to a UV light source or (ii) the THC source is only exposed to a heat source. For example, the rate constant of conversion of a THC source to CBN according to the methods of the disclosure is about 1%, about 5%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, or more, greater than the rate constant of conversion of a THC source to CBN when either (i) the THC source is only exposed to a UV light source or (ii) the THC source is only exposed to a heat source, including all ranges and subranges therebetween. In some embodiments, the rate constant of conversion of a THC source to CBN according to the methods of the disclosure is at least about 1%, at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more, greater than the rate constant of conversion of a THC source to CBN when either (i) the THC source is only exposed to a UV light source or (ii) the THC source is only exposed to a heat source, including all ranges and subranges therebetween.

In some embodiments, the rate constant of conversion of a THC source to CBN according to the methods of the disclosure (e.g., when a THC source is exposed to a UV light source, a heat source, and oxygen from an oxygen source) is between about 1% and about 500% greater than the rate constant of conversion of a THC source to CBN according to an alternative method. For example, the rate constant of conversion of a THC source to CBN according to the methods of the disclosure is about 1%, about 5%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, or more, greater than the rate constant of conversion of a THC source to CBN according to an alternative method, including all ranges and subranges therebetween. In some embodiments, the rate constant of conversion of a THC source to CBN according to the methods of the disclosure is at least about 1%, at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more, greater than the rate constant of conversion of a THC source to CBN according to an alternative method. The alternative method may be any one of the following: (i) exposing the THC source to a UV light source and a heat source; (ii) exposing the THC source to a UV light source and oxygen from an oxygen source; (iii) exposing the THC source to a heat source and oxygen from an oxygen source; (iv) exposing the THC source to a UV light source; (v) exposing the THC source to a heat source; or (vi) exposing the THC source to oxygen from an oxygen source.

In some embodiments, the rate constant of conversion of a THC source to CBN according to the methods of the disclosure (e.g., when a THC source is stirred and exposed to a UV light source, a heat source, and oxygen from an oxygen source) is between about 1% and about 500% greater than the rate constant of conversion of a THC source to CBN according to an alternative method. For example, the rate constant of conversion of a THC source to CBN according to the methods of the disclosure is about 1%, about 5%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, or more, greater than the rate constant of conversion of a THC source to CBN according to an alternative method, including all ranges and subranges therebetween. In some embodiments, the rate constant of conversion of a THC source to CBN according to the methods of the disclosure is at least about 1%, at least about 5%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, or more, greater than the rate constant of conversion of a THC source to CBN according to an alternative method. The alternative method may be any one of the following: (i) exposing the THC source to a UV light source and a heat source; (ii) exposing the THC source to a UV light source and oxygen from an oxygen source; (iii) exposing the THC source to a heat source and oxygen from an oxygen source; (iv) stirring the THC source and exposing the THC source to a UV light source; (v) stirring the THC source and exposing the THC source to a heat source; (vi) stirring the THC source and exposing the THC source to oxygen from an oxygen source; (vii) exposing the THC source to a UV light source; (viii) exposing the THC source to a heat source; (ix) exposing the THC source to oxygen from an oxygen source; (x) stirring the THC source; (xi) stirring the THC source and exposing the THC source to a heat source and oxygen from an oxygen source; (xii) stirring the THC source and exposing the THC source to a heat source and a UV light source; (xii) stirring the THC source and exposing the THC source to oxygen from an oxygen source and a UV light source; or (xiii) exposing the THC source to a heat source, a UV light source, and oxygen from an oxygen source.

In some embodiments, the methods of the disclosure have a conversion rate (e.g. the amount of THC converted to CBN per unit of time) of between about 0.1 milligrams (mg) and 50 mg per minute. For example, the methods of the disclosure have a conversion rate of about 0.1 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, about 30 mg, about 30.5 mg, about 31 mg, about 31.5 mg, about 32 mg, about 32.5 mg, about 33 mg, about 33.5 mg, about 34 mg, about 34.5 mg, about 35 mg, about 35.5 mg, about 36 mg, about 36.5 mg, about 37 mg, about 37.5 mg, about 38 mg, about 38.5 mg, about 39 mg, about 39.5 mg, about 40 mg, about 40.5 mg, about 41 mg, about 41.5 mg, about 42 mg, about 42.5 mg, about 43 mg, about 43.5 mg, about 44 mg, about 44.5 mg, about 45 mg, about 45.5 mg, about 46 mg, about 46.5 mg, about 47 mg, about 47.5 mg, about 48 mg, about 48.5 mg, about 49 mg, about 49.5 mg, or about 50 mg of THC are converted to CBN per minute, including all ranges and subranges therebetween. In some embodiments, the methods of the disclosure have a conversion rate of between about 50 mg per minute to about 300 mg per minute, between about 100 mg per minute to about 200 mg per minute, between about 130 mg to about 230 mg per minute, between about 200 mg per minute to about 300 mg per minute, between about 100 mg per minute to about 300 mg per minute, including all values and subranges therebetween. For example, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 290 mg, about 295 mg, or about 300 mg of THC are converted to CBN per minute, including all ranges and subranges therebetween.

In some embodiments, the methods minimize weight loss of a Cannabis composition. In some embodiments, the methods minimize volume loss of a Cannabis composition. In some embodiments, after completing the methods of the disclosure, less than about 40% of the volume of the initial Cannabis composition is lost. In some embodiments, after completing the methods of the disclosure, less than about 35% of the volume of the initial Cannabis composition is lost. In some embodiments, after completing the methods of the disclosure, less than about 30% of the volume of the initial Cannabis composition is lost. In some embodiments, after completing the methods of the disclosure, less than about 25% of the volume of the initial Cannabis composition is lost. In some embodiments, after completing the methods of the disclosure, less than about 20% of the volume of the initial Cannabis composition is lost. In some embodiments, after completing the methods of the disclosure, less than about 15% of the volume of the initial Cannabis composition is lost. In some embodiments, after completing the methods of the disclosure, less than about 10% of the volume of the initial Cannabis composition is lost.

In some embodiments, the Cannabis composition is placed in the container with an inside capable of holding liquid of the apparatus described in Section V of this disclosure. In some embodiments, not more than 0.5 kg of Cannabis composition per liter is placed in the container. In some embodiments, less than 0.5 kg of Cannabis composition per liter is placed in the container. In some embodiments, not more than 0.375 kg of Cannabis composition per liter is placed in the container. In some embodiments, less than 0.375 kg of Cannabis composition per liter is placed in the container. In some embodiments, the container comprises between about 0.25 kg and about 0.375 kg of Cannabis composition per liter of the container. For example, the container comprises about 0.25 kg, about 0.26 kg, about 0.27 kg, about 0.28 kg, about 0.29 kg, about 0.30 kg, about 0.31 kg, about 0.32 kg, about 0.33 kg, about 0.34 kg, about 0.35 kg, about 0.36 kg, about 0.37 kg, or about 0.375 kg of Cannabis composition per liter of the container, including all ranges and subranges therebetween. In some embodiments, the Cannabis composition occupies 30-45% of the volume of the container. For example, the Cannabis composition occupies about 30%, about 31%, about 32%, about 33%, about 34%, about 35%, about 36%, about 37%, about 38%, about 39%, about 40%, about 41%, about 42%, about 43%, about 44%, or about 45% of the volume of the container, including all subranges therebetween.

In some embodiments, a Cannabis composition is exposed to an ultraviolet (UV) light source. A UV light source produces electromagnetic radiation with a wavelength between 10 nm and 415 nm. The addition of a UV light source unexpectedly enhances the conversion of a cannabinoid, such as THC, THCA, CBD, or CBDA , to CBN.

In some embodiments, the ultraviolet light source produces electromagnetic radiation with a wavelength between about 10 nm and about 415 nm. For example, the ultraviolet light source produces electromagnetic radiation with a wavelength of about 10 nm, about 11 nm, about 12 nm, about 13 nm, about 14 nm, about 15 nm, about 16 nm, about 17 nm, about 18 nm, about 19 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, or about 415 nm, including all ranges and subranges therebetween. In some embodiments, the ultraviolet light source produces electromagnetic radiation with a wavelength of at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 22 nm, at least about 24 nm, at least about 26 nm, at least about 28 nm, at least about 30 nm, at least about 32 nm, at least about 34 nm, at least about 36 nm, at least about 38 nm, at least about 40 nm, at least about 42 nm, at least about 44 nm, at least about 46 nm, at least about 48 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, at least about 100 nm, at least about 105 nm, at least about 110 nm, at least about 115 nm, at least about 120 nm, at least about 125 nm, at least about 130 nm, at least about 135 nm, at least about 140 nm, at least about 145 nm, at least about 150 nm, at least about 155 nm, at least about 160 nm, at least about 165 nm, at least about 170 nm, at least about 175 nm, at least about 180 nm, at least about 185 nm, at least about 190 nm, at least about 195 nm, at least about 200 nm, at least about 205 nm, at least about 210 nm, at least about 215 nm, at least about 220 nm, at least about 225 nm, at least about 230 nm, at least about 235 nm, at least about 240 nm, at least about 245 nm, at least about 250 nm, at least about 255 nm, at least about 260 nm, at least about 265 nm, at least about 270 nm, at least about 275 nm, at least about 280 nm, at least about 285 nm, at least about 290 nm, at least about 295 nm, at least about 300 nm, at least about 305 nm, at least about 310 nm, at least about 315 nm, at least about 320 nm, at least about 325 nm, at least about 330 nm, at least about 335 nm, at least about 340 nm, at least about 345 nm, at least about 350 nm, at least about 355 nm, at least about 360 nm, at least about 365 nm, at least about 370 nm, at least about 375 nm, at least about 380 nm, at least about 385 nm, at least about 390 nm, at least about 395 nm, at least about 400 nm, at least about 405 nm, at least about 410 nm, or at least about 415 nm.

In some embodiments, the UV light is long-wave UV light. Long-wave UV light produces electromagnetic radiation with a wavelength between about 315 nm and 415 nm. In some embodiments, the long-wave UV light produces electromagnetic radiation with a wavelength of about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, or about 415 nm, including all ranges and subranges therebetween. In some embodiments, the long-wave UV light produces electromagnetic radiation with a wavelength of about 403 nm. In some embodiments, the long-wave UV light produces electromagnetic radiation with a wavelength of about 405 nm, including all ranges and subranges therebetween.

In some embodiments, the UV light has a wattage of between about 30 Watts (W) to about 500 W. For example, the UV light has a wattage of about 30 W, about 32 W, about 34 W, about 36 W, about 38 W, about 40 W, about 42 W, about 44 W, about 46 W, about 48 W, about 50 W, about 55 W, about 60 W, about 65 W, about 70 W, about 75 W, about 80 W, about 85 W, about 90 W, about 95 W, about 100 W, about 105 W, about 110 W, about 115 W, about 120 W, about 125 W, about 130 W, about 135 W, about 140 W, about 145 W, about 150 W, about 155 W, about 160 W, about 165 W, about 170 W, about 175 W, about 180 W, about 185 W, about 190 W, about 195 W, about 200 W, about 205 W, about 210 W, about 215 W, about 220 W, about 225 W, about 230 W, about 235 W, about 240 W, about 245 W, about 250 W, about 255 W, about 260 W, about 265 W, about 270 W, about 275 W, about 280 W, about 285 W, about 290 W, about 295 W, about 300 W, about 305 W, about 310 W, about 315 W, about 320 W, about 325 W, about 330 W, about 335 W, about 340 W, about 345 W, about 350 W, about 355 W, about 360 W, about 365 W, about 370 W, about 375 W, about 380 W, about 385 W, about 390 W, about 395 W, about 400 W, about 405 nm, about 410 nm, about 415 nm, about 420 W, about 425 W, about 430 W, about 435 W, about 440 W, about 445 W, about 450 W, about 455 W, about 460 W, about 465 W, about 470 W, about 475 W, about 480 W, about 485 W, about 490 W, about 495 W, or about 500 W, including all ranges and subranges therebetween. In some embodiments, the UV light has a wattage of at least about 30 W, at least about 32 W, at least about 34 W, at least about 36 W, at least about 38 W, at least about 40 W, at least about 42 W, at least about 44 W, at least about 46 W, at least about 48 W, at least about 50 W, at least about 55 W, at least about 60 W, at least about 65 W, at least about 70 W, at least about 75 W, at least about 80 W, at least about 85 W, at least about 90 W, at least about 95 W, at least about 100 W, at least about 105 W, at least about 110 W, at least about 115 W, at least about 120 W, at least about 125 W, at least about 130 W, at least about 135 W, at least about 140 W, at least about 145 W, at least about 150 W, at least about 155 W, at least about 160 W, at least about 165 W, at least about 170 W, at least about 175 W, at least about 180 W, at least about 185 W, at least about 190 W, at least about 195 W, at least about 200 W, at least about 205 W, at least about 210 W, at least about 215 W, at least about 220 W, at least about 225 W, at least about 230 W, at least about 235 W, at least about 240 W, at least about 245 W, at least about 250 W, at least about 255 W, at least about 260 W, at least about 265 W, at least about 270 W, at least about 275 W, at least about 280 W, at least about 285 W, at least about 290 W, at least about 295 W, at least about 300 W, at least about 305 W, at least about 310 W, at least about 315 W, at least about 320 W, at least about 325 W, at least about 330 W, at least about 335 W, at least about 340 W, at least about 345 W, at least about 350 W, at least about 355 W, at least about 360 W, at least about 365 W, at least about 370 W, at least about 375 W, at least about 380 W, at least about 385 W, at least about 390 W, at least about 395 W, at least about 400 W, at least about 405 nm, at least about 410 nm, at least about 415 nm, at least about 420 W, at least about 425 W, at least about 430 W, at least about 435 W, at least about 440 W, at least about 445 W, at least about 450 W, at least about 455 W, at least about 460 W, at least about 465 W, at least about 470 W, at least about 475 W, at least about 480 W, at least about 485 W, at least about 490 W, at least about 495 W, or at least about 500 W. In some embodiments, the UV light has a wattage of about 30 Watts (W).

In some embodiments, the Cannabis composition (e.g., THC source) is heated to a temperature of between about 100° C. and about 200° C. For example, the Cannabis composition (e.g., THC source) is heated to a temperature of about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., about 121° C., about 122° C., about 123° C., about 124° C., about 125° C., about 126° C., about 127° C., about 128° C., about 129° C., about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., about 148° C., about 149° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., about 180° C., about 181° C., about 182° C., about 183° C., about 184° C., about 185° C., about 186° C., about 187° C., about 188° C., about 189° C., about 190° C., about 191° C., about 192° C., about 193° C., about 194° C., about 195° C., about 196° C., about 197° C., about 198° C., about 199° C., or about 200° C., including all ranges and subranges therebetween. In some embodiments, the Cannabis composition (e.g., THC source) is heated to a temperature of at least about 100° C., at least about 101° C., at least about 102° C., at least about 103° C., at least about 104° C., at least about 105° C., at least about 106° C., at least about 107° C., at least about 108° C., at least about 109° C., at least about 110° C., at least about 111° C., at least about 112° C., at least about 113° C., at least about 114° C., at least about 115° C., at least about 116° C., at least about 117° C., at least about 118° C., at least about 119° C., at least about 120° C., at least about 121° C., at least about 122° C., at least about 123° C., at least about 124° C., at least about 125° C., at least about 126° C., at least about 127° C., at least about 128° C., at least about 129° C., at least about 130° C., at least about 131° C., at least about 132° C., at least about 133° C., at least about 134° C., at least about 135° C., at least about 136° C., at least about 137° C., at least about 138° C., at least about 139° C., at least about 140° C., at least about 141° C., at least about 142° C., at least about 143° C., at least about 144° C., at least about 145° C., at least about 146° C., at least about 147° C., at least about 148° C., at least about 149° C., at least about 150° C., at least about 151° C., at least about 152° C., at least about 153° C., at least about 154° C., at least about 155° C., at least about 156° C., at least about 157° C., at least about 158° C., at least about 159° C., at least about 160° C., at least about 161° C., at least about 162° C., at least about 163° C., at least about 164° C., at least about 165° C., at least about 166° C., at least about 167° C., at least about 168° C., at least about 169° C., at least about 170° C., at least about 171° C., at least about 172° C., at least about 173° C., at least about 174° C., at least about 175° C., at least about 176° C., at least about 177° C., at least about 178° C., at least about 179° C., at least about 180° C., at least about 181° C., at least about 182° C., at least about 183° C., at least about 184° C., at least about 185° C., at least about 186° C., at least about 187° C., at least about 188° C., at least about 189° C., at least about 190° C., at least about 191° C., at least about 192° C., at least about 193° C., at least about 194° C., at least about 195° C., at least about 196° C., at least about 197° C., at least about 198° C., at least about 199° C., or at least about 200° C. In some embodiments, the Cannabis composition (e.g., THC source) is heated to a temperature of at least about 100° C., at least about 110° C., at least about 120° C., or at least about 130° C. In some embodiments, the Cannabis composition (e.g., THC source) is heated to a temperature of between about 160° C. and about 180° C., between about 160° C. and about 170° C., between about 165° C. and about 175° C., between about 162° C. and about 165° C., or between about 165° C. and about 180° C. In some embodiments, exposure of a Cannabis composition to UV light in an oxygen-rich environment is conducted at a temperature of about 160° C. to about 180° C.

In some embodiments, exposure of a Cannabis composition (THC source) to UV light in an oxygen-rich environment is conducted at a temperature of between about 130° C. and about 180° C. For example, exposure of a Cannabis composition (THC source) to UV light in an oxygen-rich environment is conducted at a temperature of about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., about 148° C., about 149° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., or about 180° C., including all ranges and subranges therebetween. In some embodiments, exposure of a Cannabis composition (THC source) to UV light in an oxygen-rich environment is conducted at a temperature of at least about 130° C., at least about 131° C., at least about 132° C., at least about 133° C., at least about 134° C., at least about 135° C., at least about 136° C., at least about 137° C., at least about 138° C., at least about 139° C., at least about 140° C., at least about 141° C., at least about 142° C., at least about 143° C., at least about 144° C., at least about 145° C., at least about 146° C., at least about 147° C., at least about 148° C., at least about 149° C., at least about 150° C., at least about 151° C., at least about 152° C., at least about 153° C., at least about 154° C., at least about 155° C., at least about 156° C., at least about 157° C., at least about 158° C., at least about 159° C., at least about 160° C., at least about 161° C., at least about 162° C., at least about 163° C., at least about 164° C., at least about 165° C., at least about 166° C., at least about 167° C., at least about 168° C., at least about 169° C., at least about 170° C., at least about 171° C., at least about 172° C., at least about 173° C., at least about 174° C., at least about 175° C., at least about 176° C., at least about 177° C., at least about 178° C., at least about 179° C., or at least about 180° C. In some embodiments, exposure of a Cannabis composition to UV light in an oxygen-rich environment is conducted at a temperature of about 130° C. In some embodiments, a Cannabis composition (e.g., THC source) is exposed to UV light in an oxygen-rich environment and heated to a temperature of between about 150° C. and about 170° C., between about 160° C. and about 180° C., between about 160° C. and about 170° C., between about 165° C. and about 175° C., between about 162° C. and about 165° C., or between about 165° C. and about 180° C.

In some embodiments, exposure of a Cannabis composition to UV light in an oxygen-rich environment is conducted at a temperature of between about 150° C. and about 170° C. for example, In some embodiments, exposure of a Cannabis composition to UV light in an oxygen-rich environment is conducted at a temperature of about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., or about 170° C., including all ranges and subranges therebetween.

In some embodiments, exposure of a Cannabis composition to UV light in an oxygen-rich environment is conducted at a temperature of about 160° C.

In some embodiments, the Cannabis composition (e.g., THC source) is agitated/mixed. Persons with skill in the art will be familiar with the various methods for mixing/agitating chemical reactions. In some embodiments, the THC source is mixed by inverting, rotating, or otherwise moving the THC source. For example, in some embodiments, the THC source is placed on top of a rotary shaker. In some embodiments, the THC source is mixed via a pump flowing the THC source within the container. In some embodiments the pump removes THC source from a container, and causes it to pour back at the top, thereby mixing the THC source.

In some embodiments, the Cannabis composition (e.g., THC source) is stirred. In some embodiments, the stirring can be done by any known means for stirring. Persons with skill in the art will be familiar with ways to stir solutions. In some embodiments, the Cannabis composition is stirred via mechanized paddles, blades, arms. In some embodiments, the Cannabis composition is stirred by moving the container holding the Cannabis composition (e.g., by shaking or rotating). In some embodiments, the stirring is conducted by a magnetic mechanism capable of rotating a magnetic stir bar. In some embodiments, the stir bar is a Teflon (e.g. PTFE) magnet stir bar. In some embodiments, a magnetic stir bar is placed in the bottom center of a container containing a Cannabis composition.

In some embodiments, the Cannabis composition is stirred at a speed between about 100 rpm and about 1200 rpm. For example, the Cannabis composition is stirred at a speed of about 100 rpm, about 150 rpm, about 200 rpm, about 250 rpm, 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, about 1000 rpm, about 1050 rpm, about 1100 rpm, about 1150 rpm, or about 1200 rpm, including all values and subranges therebetween. In some embodiments, the speed of the vortex is between about 300 rpm and about 900 rpm, for example, about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, including all values and subranges therebetween. In some embodiments, the Cannabis composition is stirred at a speed of at least about 100 rpm, at least about 150 rpm, at least about 200 rpm, at least about 250 rpm, 300 rpm, at least about 350 rpm, at least about 400 rpm, at least about 450 rpm, at least about 500 rpm, at least about 550 rpm, at least about 600 rpm, at least about 650 rpm, at least about 700 rpm, at least about 750 rpm, at least about 800 rpm, at least about 850 rpm, at least about 900 rpm, at least about 950 rpm, at least about 1000 rpm, at least about 1050 rpm, at least about 1100 rpm, at least about 1150 rpm, or at least about 1200 rpm, including all values and subranges therebetween. In some embodiments, the speed of the vortex is between at least about 300 rpm and at least about 900 rpm, for example, at least about 300 rpm, at least about 350 rpm, at least about 400 rpm, at least about 450 rpm, at least about 500 rpm, at least about 550 rpm, at least about 600 rpm, at least about 650 rpm, at least about 700 rpm, at least about 750 rpm, at least about 800 rpm, at least about 850 rpm, or at least about 900 rpm. In some embodiments, the speed of the vortex is between about 550 rpm and 700 rpm. For example, the speed of the vortex is about 550 rpm, about 560 rpm, about 570 rpm, about 580 rpm, about 590 rpm, about 600 rpm, about 610 rpm, about 620 rpm, about 630 rpm, about 640 rpm, about 650 rpm, about 660 rpm, about 670 rpm, about 680 rpm, about 690 rpm, or about 700 rpm, including all values and subranges therebetween. In some embodiments, the Cannabis composition is stirred at a speed between about 400 rpm and about 800 rpm. For example, the Cannabis composition is stirred at a speed of about 400 rpm, about 410 rpm, about 420 rpm, about 430 rpm, about 440 rpm, about 450 rpm, about 460 rpm, about 470 rpm, about 480 rpm, about 490 rpm, about 500 rpm, about 510 rpm, about 520 rpm, about 530 rpm, about 540 rpm, about 550 rpm, about 560 rpm, about 570 rpm, about 580 rpm, about 590 rpm, about 600 rpm, about 610 rpm, about 620 rpm, about 630 rpm, about 640 rpm, about 650 rpm, about 660 rpm, about 670 rpm, about 680 rpm, about 690 rpm, about 700 rpm, about 710 rpm, about 720 rpm, about 730 rpm, about 740 rpm, about 750 rpm, about 760 rpm, about 770 rpm, about 780 rpm, about 790 rpm, or about 800 rpm, including all values and subranges therebetween. In some embodiments, the Cannabis composition is stirred at a sufficient speed so as to create a vortex.

In some embodiments, a Cannabis composition is exposed to UV light in an oxygen-rich environment for a time period sufficient to form CBN. In some embodiments, the time period is about 1 hour (h) to about 2 weeks (w). For example, the time period is about 1 h, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day (d), about 2 days, about 3 days, about 4 days, about 5 days, about 6 days, about 7 days, about 8 days, about 9 days, about 10 days, about 11 days, about 12 days, about 13 days, or about 14 days, including all ranges and subranges therebetween. In some embodiments, the time period is at least about 1 h, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 1 day (d), at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, the time period is less than 1 h, less than 2 hours, less than 3 hours, less than 4 hours, less than 5 hours, less than 6 hours, less than 7 hours, less than 8 hours, less than 9 hours, less than 10 hours, less than 11 hours, less than 12 hours, less than 13 hours, less than 14 hours, less than 15 hours, less than 16 hours, less than 17 hours, less than 18 hours, less than 19 hours, less than 20 hours, less than 21 hours, less than 22 hours, less than 23 hours, less than 1 day (d), less than 2 days, less than 3 days, less than 4 days, less than 5 days, less than 6 days, less than 7 days, less than 8 days, less than 9 days, less than 10 days, less than 11 days, less than 12 days, less than 13 days, or less than 14 days. In some embodiments, the time period is at least about 10 days, at least about 9 days, at least about 8 days, at least about 7 days, at least about 6 days, at least about 5 days, at least about 4 days, at least about 3 days, at least about 2 days, or at least about 1 day. In some embodiments, the time period is at least about 10 hours, at least about 9 hours, at least about 8 hours, at least about 7 hours, at least about 6 hours, at least about 5 hours, at least about 4 hours, at least about 3 hours, at least about 2 hours, or at least about 1 hour. In some embodiments, the time period is about 1 day (24 hours), about 2 days (48 hours), or about 3 days (72 hours). In some embodiments, the time period is at least about 1 day (24 hours), at least about 2 days (48 hours), or at least about 3 days (72 hours). In some embodiments, the Cannabis composition (e.g., THC source) is exposed to UV light in an oxygen-rich environment for between about 40 hours and about 60 hours, for between about 40 hours and about 50 hours, for between about 45 hours and about 50 hours, for between about 45 hours and about 55 hours, or between about 50 hours and about 60 hours. In some embodiments, the Cannabis composition (e.g., THC source) is exposed to UV light in an oxygen-rich environment for between about 40 hours to about 60 hours. For example, Cannabis composition (e.g., THC source) is exposed to UV light in an oxygen-rich environment for about 40 hours, about 41 hours, about 42 hours, about 43 hours, about 44 hours, about 45 hours, about 46 hours, about 47 hours, about 48 hours (2 days), about 49 hours, about 50 hours, about 51 hours, about 52 hours, about 53 hours, about 54 hours, about 55 hours, about 55 hours, about 56 hours, about 57 hours, about 58 hours, about 59 hours, or about 60 hours, including all ranges and subranges therebetween.

In some embodiments, the UV light is placed within about 5 inches of the Cannabis composition. For example, the UV light is placed within about 5 inches, within about 4.9 inches, within about 4.8 inches, within about 4.7 inches, within about 4.6 inches, within about 4.5 inches, within about 4.4 inches, within about 4.3 inches, within about 4.2 inches, within about 4.1 inches, within 4 inches, within about 3.9 inches, within about 3.8 inches, within about 3.7 inches, within about 3.6 inches, within about 3.5 inches, within about 3.4 inches, within about 3.3 inches, within about 3.2 inches, within about 3.1 inches, within about 3 inches, within about 2.9 inches, within about 2.8 inches, within about 2.7 inches, within about 2.6 inches, within about 2.5 inches, within about 2.4 inches, within about 2.3 inches, within about 2.2 inches, within about 2.1 inches, within about 2 inches, within about 1.9 inches, within about 1.8 inches, within about 1.7 inches, within about 1.6 inches, within about 1.5 inches, within about 1.4 inches, within about 1.3 inches, within about 1.2 inches, within about 1.1 inches, within about 1 inch, within about 0.9 inches, within about 0.8 inches, within about 0.7 inches, within about 0.6 inches, within about 0.5 inches, within about 0.4 inches, within about 0.3 inches, within about 0.2 inches, or within about 0.1 inches of the Cannabis composition, including all ranges and subranges therebetween. In some embodiments, the UV light is placed between about 0.1 inches and about 5 inches, between about 1 inches and about 5 inches, between about 2 inches and about 5 inches, between about 3 inches and about 5 inches, between about 4 inches and about 5 inches, between about 0.1 inches and about 4 inches, between about 1 inches and about 4 inches, between about 2 inches and about 4 inches, between about 3 inches and about 4 inches, between about 0.1 inches and about 3 inches, between about 1 inches and about 3 inches, between about 2 inches and about 3 inches, between about 0.1 inches and about 2 inches, between about 1 inches and about 2 inches, or between about 0.1 inch and about 1 inch from the Cannabis composition.

In some embodiments, an oxygen-rich environment is provided by introducing oxygen into the Cannabis composition. Persons having skill in the art will be familiar with how to oxygenate solutions. In some embodiments, the present methods are compatible with any method of introducing oxygen into a solution. In some embodiments, the oxygen-rich environment occurs by allowing gas exchange between the Cannabis composition and atmospheric air. In some embodiments, the oxygen rich environment is achieved by aerating the solution or bubbling it (e.g., by blowing oxygen into the Cannabis composition). In some embodiments, the oxygen-rich environment comprises introducing oxygen at a higher concentration than is present in the atmosphere (e.g., via an enhanced oxygen system). In some embodiments, oxygen is provided using an enhanced oxygen system. In some embodiments, the enhanced oxygen system increases oxygen injection from 10% by weight to up to 60% by weight.

In some embodiments, the oxygen is introduced into a top layer of a liquid. A top layer of a liquid is the top 50% by volume of a container containing the liquid. In some embodiments, oxygen is introduced into the top 50%, or top 40%, or top 30%, or top 20%, or top 10%, or top 5% of a container comprising a Cannabis composition. In some embodiments, oxygen is injected into the top 1 inch to 2 inches of a container containing a Cannabis composition.

### V. Apparatus for the Production of CBN

In some embodiments, the disclosure provides an apparatus for converting a cannabinoid to CBN. In some embodiments, the apparatus comprises (a) an ultraviolet (UV) light source; (b) an oxygen source with an opening through which oxygen is released; (c) a container with an inside capable of holding liquid; and (d) a heat source; wherein the UV source is located so as to shine UV light inside the container; wherein the oxygen source provides oxygen to the inside of the container; and wherein the heat source is located so as to be able to heat the container. In some embodiments, an apparatus disclosed herein is utilized to accomplish the methods of the disclosure.

In some embodiments, the apparatus contains a UV light source. Ultraviolet (UV) is a form of electromagnetic radiation with wavelength from 10 nm (with a corresponding frequency around 30 PHz) to 405 nm (750 THz). UV light is shorter than that of visible light, but longer than X-rays. UV radiation is present in sunlight, and constitutes about 10% of the total electromagnetic radiation output from the Sun. It is also produced by electric arcs and specialized lights, such as mercury-vapor lamps, tanning lamps, and black lights.

For the purposes of this disclosure, the UV spectrum can be categorized as shown in Table A, below:

TABLE A
UV spectrum

| | | Wavelength | Photon energy |
|---|---|---|---|
| Name | Abbreviation | (nm) | (eV, aJ) |
| UltravioletA | UV-A | 315-415 | 3.10-3.94, |
| (Long-wave) | | | 0.497-0.631 |
| Ultraviolet B | UV-B | 280-315 | 3.94-4.43, |
| (Medium-wave) | | | 0.631-0.710 |
| Ultraviolet C | UV-C | 100-280 | 4.43-12.4, |
| (Short-wave) | | | 0.710-1.987 |

Persons having skill in the art will be familiar with the various sources of UV light, and will be able to pick the most appropriate light source for the desired UV light emission. A non-limiting list of UV light emitting devices is provided in Table B, below.

TABLE B
UV Light Emitters

| Name | Devices |
|---|---|
| Ultraviolet A | Black light |
| (Long-wave) | Light-emitting diodes |
| | Gas lasers |
| | Laser diodes |
| | Solid-state lasers |
| | UV-A lamps |
| Ultraviolet B | Gas lasers |
| (Medium-wave) | Laser diodes |
| | Solid-state lasers |
| | UV-B lamps |
| | Light-emitting diodes |
| Ultraviolet C | Short-wave ultraviolet lamp |
| (Short-wave) | Light-emitting diodes |
| | Gas lasers |
| | Laser diodes |
| | Solid-state lasers |
| | Low-pressure mercury lamp |
| | Excimer lamp |
| | Pulsed xenon lamps |

Persons having skill in the art will understand that a large number of UV-emitting devices emit across a spectrum of UV radiation. In some embodiments, a UV light may have its peak emissions at the “long-wave” UV spectrum, but may also have some emissions at higher or lower wavelengths. In some embodiments, the methods and devices of the present disclosure utilize narrow spectrum emitters and/or narrow band light. That is, in some embodiments, the UV light source emits at a narrow range, such that its emissions span less than 100 nm, 75 nm, 50 nm, or 25 nm of total spectrum range (e.g. 300-400 nm). In some embodiments, the UV light source emits a broad spectrum of electromagnetic radiation, spanning more than 100 nm of total spectrum range. In some embodiments the UV light source emits electromagnetic radiation at multiple peaks across the spectrum.

In some embodiments, the UV light emitting device is a black light, a light-emitting diode, a gas laser, a laser diode, a solid-state laser, a UV-A lamp, a UV-B lamp, a UV-C lamp, a short-wave ultraviolet lamp, a low-pressure mercury lamp, an excimer lamp, a pulsed xenon lamp, a gas discharge lamp, a xenon arc lamp, a deuterium arc lamp, a mercury-xenon arc lamp, or a metal-halide arc lamp.

In some embodiments, the UV light source produces electromagnetic radiation with a wavelength between about 10 nm and about 415 nm. For example, the UV light source produces electromagnetic radiation with a wavelength of about 10 nm, about 11 nm, about 12 nm, about 13 nm, about 14 nm, about 15 nm, about 16 nm, about 17 nm, about 18 nm, about 19 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, or about 415 nm, including any ranges and subranges therebetween. In some embodiments, the UV light source produces electromagnetic radiation with a wavelength of at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 22 nm, at least about 24 nm, at least about 26 nm, at least about 28 nm, at least about 30 nm, at least about 32 nm, at least about 34 nm, at least about 36 nm, at least about 38 nm, at least about 40 nm, at least about 42 nm, at least about 44 nm, at least about 46 nm, at least about 48 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, at least about 100 nm, at least about 105 nm, at least about 110 nm, at least about 115 nm, at least about 120 nm, at least about 125 nm, at least about 130 nm, at least about 135 nm, at least about 140 nm, at least about 145 nm, at least about 150 nm, at least about 155 nm, at least about 160 nm, at least about 165 nm, at least about 170 nm, at least about 175 nm, at least about 180 nm, at least about 185 nm, at least about 190 nm, at least about 195 nm, at least about 200 nm, at least about 205 nm, at least about 210 nm, at least about 215 nm, at least about 220 nm, at least about 225 nm, at least about 230 nm, at least about 235 nm, at least about 240 nm, at least about 245 nm, at least about 250 nm, at least about 255 nm, at least about 260 nm, at least about 265 nm, at least about 270 nm, at least about 275 nm, at least about 280 nm, at least about 285 nm, at least about 290 nm, at least about 295 nm, at least about 300 nm, at least about 305 nm, at least about 310 nm, at least about 315 nm, at least about 320 nm, at least about 325 nm, at least about 330 nm, at least about 335 nm, at least about 340 nm, at least about 345 nm, at least about 350 nm, at least about 355 nm, at least about 360 nm, at least about 365 nm, at least about 370 nm, at least about 375 nm, at least about 380 nm, at least about 385 nm, at least about 390 nm, at least about 395 nm, at least about 400 nm, at least about 405 nm, at least about 410 nm, or at least about 415 nm.

In some embodiments, the UV light source produces long-wave UV light. In some embodiments, the long-wave UV light produces electromagnetic radiation with a wavelength of about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, or about 415 nm, including any ranges and subranges therebetween. In some embodiments, the long-wave UV light produces electromagnetic radiation with a wavelength of about 403 nm. In some embodiments, the long-wave UV light produces electromagnetic radiation with a wavelength of about 405 nm.

In some embodiments, the UV light source has a wattage of between about 30 Watts (W) to about 500 W. For example, the UV light source has a wattage of about 30 W, about 32 W, about 34 W, about 36 W, about 38 W, about 40 W, about 42 W, about 44 W, about 46 W, about 48 W, about 50 W, about 55 W, about 60 W, about 65 W, about 70 W, about 75 W, about 80 W, about 85 W, about 90 W, about 95 W, about 100 W, about 105 W, about 110 W, about 115 W, about 120 W, about 125 W, about 130 W, about 135 W, about 140 W, about 145 W, about 150 W, about 155 W, about 160 W, about 165 W, about 170 W, about 175 W, about 180 W, about 185 W, about 190 W, about 195 W, about 200 W, about 205 W, about 210 W, about 215 W, about 220 W, about 225 W, about 230 W, about 235 W, about 240 W, about 245 W, about 250 W, about 255 W, about 260 W, about 265 W, about 270 W, about 275 W, about 280 W, about 285 W, about 290 W, about 295 W, about 300 W, about 305 W, about 310 W, about 315 W, about 320 W, about 325 W, about 330 W, about 335 W, about 340 W, about 345 W, about 350 W, about 355 W, about 360 W, about 365 W, about 370 W, about 375 W, about 380 W, about 385 W, about 390 W, about 395 W, about 400 W, about 405 nm, about 410 nm, about 415 nm, about 420 W, about 425 W, about 430 W, about 435 W, about 440 W, about 445 W, about 450 W, about 455 W, about 460 W, about 465 W, about 470 W, about 475 W, about 480 W, about 485 W, about 490 W, about 495 W, or about 500 W, including any subranges and ranges therebetween. In some embodiments, the UV light source has a wattage of at least about 30 W, at least about 32 W, at least about 34 W, at least about 36 W, at least about 38 W, at least about 40 W, at least about 42 W, at least about 44 W, at least about 46 W, at least about 48 W, at least about 50 W, at least about 55 W, at least about 60 W, at least about 65 W, at least about 70 W, at least about 75 W, at least about 80 W, at least about 85 W, at least about 90 W, at least about 95 W, at least about 100 W, at least about 105 W, at least about 110 W, at least about 115 W, at least about 120 W, at least about 125 W, at least about 130 W, at least about 135 W, at least about 140 W, at least about 145 W, at least about 150 W, at least about 155 W, at least about 160 W, at least about 165 W, at least about 170 W, at least about 175 W, at least about 180 W, at least about 185 W, at least about 190 W, at least about 195 W, at least about 200 W, at least about 205 W, at least about 210 W, at least about 215 W, at least about 220 W, at least about 225 W, at least about 230 W, at least about 235 W, at least about 240 W, at least about 245 W, at least about 250 W, at least about 255 W, at least about 260 W, at least about 265 W, at least about 270 W, at least about 275 W, at least about 280 W, at least about 285 W, at least about 290 W, at least about 295 W, at least about 300 W, at least about 305 W, at least about 310 W, at least about 315 W, at least about 320 W, at least about 325 W, at least about 330 W, at least about 335 W, at least about 340 W, at least about 345 W, at least about 350 W, at least about 355 W, at least about 360 W, at least about 365 W, at least about 370 W, at least about 375 W, at least about 380 W, at least about 385 W, at least about 390 W, at least about 395 W, at least about 400 W, at least about 405 nm, at least about 410 nm, at least about 415 nm, at least about 420 W, at least about 425 W, at least about 430 W, at least about 435 W, at least about 440 W, at least about 445 W, at least about 450 W, at least about 455 W, at least about 460 W, at least about 465 W, at least about 470 W, at least about 475 W, at least about 480 W, at least about 485 W, at least about 490 W, at least about 495 W, or at least about 500 W. In some embodiments, the UV light source has a wattage of about 30 Watts (W). In some embodiments, larger industrial-scale conversion apparatuses will have larger wattage light sources.

In some embodiments, the UV light source has an irradiance measured in, for example, watts of energy per meter squared of surface area (W/m²). In some embodiments, the UV light source has an irradiance at the UV-A range of about 0.1 W/m², about 0.25 W/m², about 0.5 W/m², about 0.75 W/m², about 1 W/m², about 10 W/m², about 20 W/m², about 30 W/m², about 40 W/m², about 50 W/m², about 60 W/m², about 70 W/m², about 80 W/m², about 90 W/m², about 100 W/m², about 110 W/m², about 120 W/m², about 130 W/m², about 140 W/m², about 150 W/m², about 160 W/m², about 170 W/m², about 180 W/m², about 190 W/m², about 200 W/m², about 210 W/m², about 220 W/m², about 230 W/m², about 240 W/m², about 250 W/m², about 260 W/m², about 270 W/m², about 280 W/m², about 290 W/m², about 300 W/m², about 310 W/m², about 320 W/m², about 330 W/m², about 340 W/m², about 350 W/m², about 360 W/m², about 370 W/m², about 380 W/m², about 390 W/m², about 400 W/m², about 410 W/m², about 420 W/m², about 430 W/m², about 440 W/m², about 450 W/m², about 460 W/m², about 470 W/m², about 480 W/m², about 490 W/m², about 500 W/m², about 510 W/m², about 520 W/m², about 530 W/m², 540 W/m², 550 W/m², about 560 W/m², about 570 W/m², about 580 W/m², about 590 W/m², about 600 W/m², about 610 W/m², about 620 W/m², about 630 W/m², about 640 W/m², about 650 W/m², about 660 W/m², about 670 W/m², about 680 W/m², about 690 W/m², about 700 W/m², about 710 W/m², about 720 W/m², about 730 W/m², about 740 W/m², about 750 W/m², about 760 W/m², about 770 W/m², about 780 W/m², about 790 W/m², about 800 W/m², about 810 W/m², about 820 W/m², about 830 W/m², about 840 W/m², about 850 W/m², about 860 W/m², about 870 W/m², about 880 W/m², about 890 W/m², about 900 W/m², about 910 W/m², about 920 W/m², about 930 W/m², about 940 W/m², about 950 W/m², about 960 W/m², about 970 W/m², about 980 W/m², about 990 W/m², about 1000 W/m², about 1250 W/m², about 1500 W/m², about 1750 W/m², about 2000 W/m², about 2250 W/m², about 2500 W/m², about 2750 W/m², about 3000 W/m², about 3250 W/m², about 3500 W/m², about 3750 W/m², about 4000 W/m², including any subranges and ranges therebetween. In some embodiments, the UV light source has an irradiance at the UV-A range of at least about 0.1 W/m², at least about 0.25 W/m², at least about 0.5 W/m², at least about 0.75 W/m², at least about 1 W/m², at least about 10 W/m², at least about 20 W/m², at least about 30 W/m², at least about 40 W/m², at least about 50 W/m², at least about 60 W/m², at least about 70 W/m², at least about 80 W/m², at least about 90 W/m², at least about 100 W/m², at least about 110 W/m², at least about 120 W/m², at least about 130 W/m², at least about 140 W/m², at least about 150 W/m², at least about 160 W/m², at least about 170 W/m², at least about 180 W/m², at least about 190 W/m², at least about 200 W/m², at least about 210 W/m², at least about 220 W/m², at least about 230 W/m², at least about 240 W/m², at least about 250 W/m², at least about 260 W/m², at least about 270 W/m², at least about 280 W/m², at least about 290 W/m², at least about 300 W/m², at least about 310 W/m², at least about 320 W/m², at least about 330 W/m², at least about 340 W/m², at least about 350 W/m², at least about 360 W/m², at least about 370 W/m², at least about 380 W/m², at least about 390 W/m², at least about 400 W/m², at least about 410 W/m², at least about 420 W/m², at least about 430 W/m², at least about 440 W/m², at least about 450 W/m², at least about 460 W/m², at least about 470 W/m², at least about 480 W/m², at least about 490 W/m², at least about 500 W/m², at least about 510 W/m², at least about 520 W/m², at least about 530 W/m², 540 W/m², 550 W/m², at least about 560 W/m², at least about 570 W/m², at least about 580 W/m², at least about 590 W/m², at least about 600 W/m², at least about 610 W/m², at least about 620 W/m², at least about 630 W/m², at least about 640 W/m², at least about 650 W/m², at least about 660 W/m², at least about 670 W/m², at least about 680 W/m², at least about 690 W/m², at least about 700 W/m², at least about 710 W/m², at least about 720 W/m², at least about 730 W/m², at least about 740 W/m², at least about 750 W/m², at least about 760 W/m², at least about 770 W/m², at least about 780 W/m², at least about 790 W/m², at least about 800 W/m², at least about 810 W/m², at least about 820 W/m², at least about 830 W/m², at least about 840 W/m², at least about 850 W/m², at least about 860 W/m², at least about 870 W/m², at least about 880 W/m², at least about 890 W/m², at least about 900 W/m², at least about 910 W/m², at least about 920 W/m², at least about 930 W/m², at least about 940 W/m², at least about 950 W/m², at least about 960 W/m², at least about 970 W/m², at least about 980 W/m², at least about 990 W/m², at least about 1000 W/m², at least about 1250 W/m², at least about 1500 W/m², at least about 1750 W/m², at least about 2000 W/m², at least about 2250 W/m², at least about 2500 W/m², at least about 2750 W/m², at least about 3000 W/m², at least about 3250 W/m², at least about 3500 W/m², at least about 3750 W/m², at least about 4000 W/m². In some embodiments, the UV light source has an irradiance at the UV-A range of at least about 10 W/m².In some embodiments, the apparatus comprises an oxygen source with an opening through which oxygen is provided to the container. In some embodiments, the present methods and devices of the present disclosure are compatible with any oxygen source. Persons having skill in the art will be familiar with various oxygen sources. In embodiments, the oxygen source comprises exogenous oxygen. In some embodiments, the oxygen source is an oxygen tank. Pure or highly concentrated oxygen is commercially available in a variety of containers, ranging from small spray cans to large M2 to M250 sizes. In some embodiments, the oxygen tank comprises oxygen. In some embodiments, in addition to oxygen, the oxygen tank contains other gases, including helium, argon, carbon dioxide, carbon monoxide, or any combination thereof. In some embodiments, the oxygen tank is a compressed oxygen cylinder. In some embodiments, the oxygen source is an oxygen concentrator. In some embodiments oxygen concentrators compress atmospheric air, remove nitrogen via e.g., a sieve bed filter, and then releases the air at desired oxygen concentrations. Oxygen concentrators range from large commercial concentrators, such as those used to fill scuba tanks, oxygenate aquarium fish tanks, or coal gasification, to small portable units carried by patients requiring enriched oxygen treatments. The particulars of various oxygen concentrators are well known in the art, and are therefore not reviewed further in this document.

. In some embodiments oxygen source delivers air enriched in oxygen through an opening. In some embodiments, the diameter of the opening through which oxygen is injected is between about 0.1 p.m and about 1 cm. In some embodiments, the diameter of the opening is 0.5 microns. In some embodiments, the oxygen source provides gas between about 10% and about 100% oxygen by weight. For example, the oxygen source provides gas of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% oxygen by weight, including all ranges and subranges therebetween. In some embodiments, the oxygen source provides gas of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% oxygen by weight. In some embodiments, the oxygen source provides gas with an oxygen content of at least 21% by weight. In some embodiments, the oxygen sources provides a gas comprising between about 10% and about 30% oxygen by weight. For example, the oxygen source provides gas of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, including all values and subranges therebetween.

In some embodiments, the oxygen source provides a gas enriched in oxygen over atmospheric air. In some embodiments, the oxygen source provides gas comprising between about 10% and about 30% oxygen more by weight than atmospheric air. For example, the oxygen source provides gas comprising about 10% increased oxygen (about 31% absolute oxygen by weight), about 11% (about 32% absolute oxygen by weight), about 12% (about 33% absolute oxygen by weight), about 13% (about 34% absolute oxygen by weight), about 14% (about 35% absolute oxygen by weight), about 15% (about 36% absolute oxygen by weight), about 16% (about 37% absolute oxygen by weight), about 17% (about 38% absolute oxygen by weight), about 18% (about 39% absolute oxygen by weight), about 19% (about 40% absolute oxygen by weight), about 20% (about 41% absolute oxygen by weight), about 21% (about 42% absolute oxygen by weight), about 22% (about 43% absolute oxygen by weight), about 23% (about 44% absolute oxygen by weight), about 24% (about 45% absolute oxygen by weight), about 25% (about 46% absolute oxygen by weight), about 26% (about 47% absolute oxygen by weight), about 27% (about 48% absolute oxygen by weight), about 28% (about 49% absolute oxygen by weight), about 29% (about 50% absolute oxygen by weight), or about 30% (about 51% absolute oxygen by weight), including all values and subranges therebetween. For example, the oxygen source provides gas comprising about 10% increased oxygen (about 31% absolute oxygen by weight), about 11% (about 32% absolute oxygen by weight), about 12% (about 33% absolute oxygen by weight), about 13% (about 34% absolute oxygen by weight), about 14% (about 35% absolute oxygen by weight), about 15% (about 36% absolute oxygen by weight), about 16% (about 37% absolute oxygen by weight), about 17% (about 38% absolute oxygen by weight), about 18% (about 39% absolute oxygen by weight), about 19% (about 40% absolute oxygen by weight), about 20% (about 41% absolute oxygen by weight), about 21% (about 42% absolute oxygen by weight), about 22% (about 43% absolute oxygen by weight), about 23% (about 44% absolute oxygen by weight), about 24% (about 45% absolute oxygen by weight), about 25% (about 46% absolute oxygen by weight), about 26% (about 47% absolute oxygen by weight), about 27% (about 48% absolute oxygen by weight), about 28% (about 49% absolute oxygen by weight), about 29% (about 50% absolute oxygen by weight), or about 30% (about 51% absolute oxygen by weight), including all values and subranges therebetween.

In some embodiments the oxygen source provides gas that is enriched by at least about 10% (about 31% absolute oxygen by weight), about 11% (about 32% absolute oxygen by weight), about 12% (about 33% absolute oxygen by weight), about 13% (about 34% absolute oxygen by weight), about 14% (about 35% absolute oxygen by weight), about 15% (about 36% absolute oxygen by weight), about 16% (about 37% absolute oxygen by weight), about 17% (about 38% absolute oxygen by weight), about 18% (about 39% absolute oxygen by weight), about 19% (about 40% absolute oxygen by weight), about 20% (about 41% absolute oxygen by weight), about 21% (about 42% absolute oxygen by weight), about 22% (about 43% absolute oxygen by weight), about 23% (about 44% absolute oxygen by weight), about 24% (about 45% absolute oxygen by weight), about 25% (about 46% absolute oxygen by weight), about 26% (about 47% absolute oxygen by weight), about 27% (about 48% absolute oxygen by weight), about 28% (about 49% absolute oxygen by weight), about 29% (about 50% absolute oxygen by weight), or about 30% (about 51% absolute oxygen by weight), including all values and subranges therebetween.

In some embodiments, the oxygen source is an air pump that increases the flow of atmospheric oxygen to the THC source. That is in some embodiments, exogenous oxygen is atmospheric oxygen that is artificially pumped into the THC source while THC is being converted to CBN.

In some embodiments, the oxygen source provides gas at a flow rate of between about 1 milliliter per minute (mL/min) and 100 liters per minute (L/min). For example, the oxygen source provides gas at a flow rate of about 1 mL/min, about 2 mL/min, about 1 mL/min, about 2 mL/min, about 3 mL/min, about 4 mL/min, about 5 mL/min, about 6 mL/min, about 7 mL/min, about 8 mL/min, about 9 mL/min, about 10 mL/min, about 11 mL/min, about 12 mL/min, about 13 mL/min, about 14 mL/min, about 15 mL/min, about 16 mL/min, about 17 mL/min, about 18 mL/min, about 19 mL/min, about 20 mL/min, about 21 mL/min, about 22 mL/min, about 23 mL/min, about 24 mL/min, about 25 mL/min, about 26 mL/min, about 27 mL/min, about 28 mL/min, about 29 mL/min, about 30 mL/min, about 31 mL/min, about 32 mL/min, about 33 mL/min, about 34 mL/min, about 35 mL/min, about 36 mL/min, about 37 mL/min, about 38 mL/min, about 39 mL/min, about 40 mL/min, about 41 mL/min, about 42 mL/min, about 43 mL/min, about 44 mL/min, about 45 mL/min, about 46 mL/min, about 47 mL/min, about 48 mL/min, about 49 mL/min, about 50 mL/min, about 51 mL/min, about 52 mL/min, about 53 mL/min, about 54 mL/min, about 55 mL/min, about 56 mL/min, about 57 mL/min, about 58 mL/min, about 59 mL/min, about 60 mL/min, about 61 mL/min, about 62 mL/min, about 63 mL/min, about 64 mL/min, about 65 mL/min, about 66 mL/min, about 67 mL/min, about 68 mL/min, about 69 mL/min, about 70 mL/min, about 71 mL/min, about 72 mL/min, about 73 mL/min, about 74 mL/min, about 75 mL/min, about 76 mL/min, about 77 mL/min, about 78 mL/min, about 79 mL/min, about 80 mL/min, about 81 mL/min, about 82 mL/min, about 83 mL/min, about 84 mL/min, about 85 mL/min, about 86 mL/min, about 87 mL/min, about 88 mL/min, about 89 mL/min, about 90 mL/min, about 91 mL/min, about 92 mL/min, about 93 mL/min, about 94 mL/min, about 95 mL/min, about 96 mL/min, about 97 mL/min, about 98 mL/min, about 99 mL/min, about 100 mL/min, about 105 mL/min, about 110 mL/min, about 115 mL/min, about 120 mL/min, about 125 mL/min, about 130 mL/min, about 135 mL/min, about 140 mL/min, about 145 mL/min, about 150 mL/min, about 155 mL/min, about 160 mL/min, about 165 mL/min, about 170 mL/min, about 175 mL/min, about 180 mL/min, about 185 mL/min, about 190 mL/min, about 195 mL/min, about 200 mL/min, about 225 mL/min, about 250 mL/min, about 275 mL/min, about 300 mL/min, about 325 mL/min, about 350 mL/min, about 375 mL/min, about 400 mL/min, about 425 mL/min, about 450 mL/min, about 475 mL/min, about 500 mL/min, about 525 mL/min, about 550 mL/min, about 575 mL/min, about 600 mL/min, about 625 mL/min, about 650 mL/min, about 675 mL/min, about 700 mL/min, about 725 mL/min, about 750 mL/min, about 775 mL/min, about 800 mL/min, about 825 mL/min, about 850 mL/min, about 875 mL/min, about 900 mL/min, about 925 mL/min, about 950 mL/min, about 975 mL/min, about 1 L/min, about 1.25 L/min, about 1.5 L/min, about 1.75 L/min, about 2 L/min, about 2.25 L/min, about 2.5 L/min, about 2.75 L/min, about 3 L/min, about 3.25 L/min, about 3.5 L/min, about 3.75 L/min, about 4 L/min, about 4.25 L/min, about 4.5 L/min, about 4.75 L/min, about 5 L/min, about 5.25 L/min, about 5.5 L/min, about 5.75 L/min, about 6 L/min, about 6.25 L/min, about 6.5 L/min, about 6.75 L/min, about 7 L/min, about 7.25 L/min, about 7.5 L/min, about 7.75 L/min, about 8 L/min, about 8.25 L/min, about 8.5 L/min, about 8.75 L/min, about 9 L/min, about 9.25 L/min, about 9.5 L/min, about 9.75 L/min, about 10 L/min, about 10.25 L/min, about 10.5 L/min, about 10.75 L/min, about 11 L/min, about 11.25 L/min, about 11.5 L/min, about 11.75 L/min, about 12 L/min, about 12.25 L/min, about 12.5 L/min, about 12.75 L/min, about 13 L/min, about 13.25 L/min, about 13.5 L/min, about 13.75 L/min, about 14 L/min, about 14.25 L/min, about 14.5 L/min, about 14.75 L/min, about 15 L/min, about 15.25 L/min, about 15.5 L/min, about 15.75 L/min, about 16 L/min, about 16.25 L/min, about 16.5 L/min, about 16.75 L/min, about 17 L/min, about 17.25 L/min, about 17.5 L/min, about 17.75 L/min, about 18 L/min, about 18.25 L/min, about 18.5 L/min, about 18.75 L/min, about 19 L/min, about 19.25 L/min, about 19.5 L/min, about 19.75 L/min, about 20 L/min, about 20.25 L/min, about 20.5 L/min, about 20.75 L/min, about 21 L/min, about 21.25 L/min, about 21.5 L/min, about 21.75 L/min, about 22 L/min, about 22.25 L/min, about 22.5 L/min, about 22.75 L/min, about 23 L/min, about 23.25 L/min, about 23.5 L/min, about 23.75 L/min, about 24 L/min, about 24.25 L/min, about 24.5 L/min, about 24.75 L/min, about 25 L/min, about 25.25 L/min, about 25.5 L/min, about 25.75 L/min, about 26 L/min, about 26.25 L/min, about 26.5 L/min, about 26.75 L/min, about 27 L/min, about 27.25 L/min, about 27.5 L/min, about 27.75 L/min, about 28 L/min, about 28.25 L/min, about 28.5 L/min, about 28.75 L/min, about 29 L/min, about 29.25 L/min, about 29.5 L/min, about 29.75 L/min, about 30 L/min, about 30.25 L/min, about 30.5 L/min, about 30.75 L/min, about 31 L/min, about 31.25 L/min, about 31.5 L/min, about 31.75 L/min, about 32 L/min, about 32.25 L/min, about 32.5 L/min, about 32.75 L/min, about 33 L/min, about 33.25 L/min, about 33.5 L/min, about 33.75 L/min, about 34 L/min, about 34.25 L/min, about 34.5 L/min, about 34.75 L/min, about 35 L/min, about 35.25 L/min, about 35.5 L/min, about 35.75 L/min, about 36 L/min, about 36.25 L/min, about 36.5 L/min, about 36.75 L/min, about 37 L/min, about 37.25 L/min, about 37.5 L/min, about 37.75 L/min, about 38 L/min, about 38.25 L/min, about 38.5 L/min, about 38.75 L/min, about 39 L/min, about 39.25 L/min, about 39.5 L/min, about 39.75 L/min, about 40 L/min, about 40.25 L/min, about 40.5 L/min, about 40.75 L/min, about 41 L/min, about 41.25 L/min, about 41.5 L/min, about 41.75 L/min, about 42 L/min, about 42.25 L/min, about 42.5 L/min, about 42.75 L/min, about 43 L/min, about 43.25 L/min, about 43.5 L/min, about 43.75 L/min, about 44 L/min, about 44.25 L/min, about 44.5 L/min, about 44.75 L/min, about 45 L/min, about 45.25 L/min, about 45.5 L/min, about 45.75 L/min, about 46 L/min, about 46.25 L/min, about 46.5 L/min, about 46.75 L/min, about 47 L/min, about 47.25 L/min, about 47.5 L/min, about 47.75 L/min, about 48 L/min, about 48.25 L/min, about 48.5 L/min, about 48.75 L/min, about 49 L/min, about 49.25 L/min, about 49.5 L/min, about 49.75 L/min, about 50 L/min, about 50.25 L/min, about 50.5 L/min, about 50.75 L/min, about 51 L/min, about 51.25 L/min, about 51.5 L/min, about 51.75 L/min, about 52 L/min, about 52.25 L/min, about 52.5 L/min, about 52.75 L/min, about 53 L/min, about 53.25 L/min, about 53.5 L/min, about 53.75 L/min, about 54 L/min, about 54.25 L/min, about 54.5 L/min, about 54.75 L/min, about 55 L/min, about 55.25 L/min, about 55.5 L/min, about 55.75 L/min, about 56 L/min, about 56.25 L/min, about 56.5 L/min, about 56.75 L/min, about 57 L/min, about 57.25 L/min, about 57.5 L/min, about 57.75 L/min, about 58 L/min, about 58.25 L/min, about 58.5 L/min, about 58.75 L/min, about 59 L/min, about 59.25 L/min, about 59.5 L/min, about 59.75 L/min, about 60 L/min, about 60.25 L/min, about 60.5 L/min, about 60.75 L/min, about 61 L/min, about 61.25 L/min, about 61.5 L/min, about 61.75 L/min, about 62 L/min, about 62.25 L/min, about 62.5 L/min, about 62.75 L/min, about 63 L/min, about 63.25 L/min, about 63.5 L/min, about 63.75 L/min, about 64 L/min, about 64.25 L/min, about 64.5 L/min, about 64.75 L/min, about 65 L/min, about 65.25 L/min, about 65.5 L/min, about 65.75 L/min, about 66 L/min, about 66.25 L/min, about 66.5 L/min, about 66.75 L/min, about 67 L/min, about 67.25 L/min, about 67.5 L/min, about 67.75 L/min, about 68 L/min, about 68.25 L/min, about 68.5 L/min, about 68.75 L/min, about 69 L/min, about 69.25 L/min, about 69.5 L/min, about 69.75 L/min, about 70 L/min, about 70.25 L/min, about 70.5 L/min, about 70.75 L/min, about 71 L/min, about 71.25 L/min, about 71.5 L/min, about 71.75 L/min, about 72 L/min, about 72.25 L/min, about 72.5 L/min, about 72.75 L/min, about 73 L/min, about 73.25 L/min, about 73.5 L/min, about 73.75 L/min, about 74 L/min, about 74.25 L/min, about 74.5 L/min, about 74.75 L/min, about 75 L/min, about 75.25 L/min, about 75.5 L/min, about 75.75 L/min, about 76 L/min, about 76.25 L/min, about 76.5 L/min, about 76.75 L/min, about 77 L/min, about 77.25 L/min, about 77.5 L/min, about 77.75 L/min, about 78 L/min, about 78.25 L/min, about 78.5 L/min, about 78.75 L/min, about 79 L/min, about 79.25 L/min, about 79.5 L/min, about 79.75 L/min, about 80 L/min, about 80.25 L/min, about 80.5 L/min, about 80.75 L/min, about 81 L/min, about 81.25 L/min, about 81.5 L/min, about 81.75 L/min, about 82 L/min, about 82.25 L/min, about 82.5 L/min, about 82.75 L/min, about 83 L/min, about 83.25 L/min, about 83.5 L/min, about 83.75 L/min, about 84 L/min, about 84.25 L/min, about 84.5 L/min, about 84.75 L/min, about 85 L/min, about 85.25 L/min, about 85.5 L/min, about 85.75 L/min, about 86 L/min, about 86.25 L/min, about 86.5 L/min, about 86.75 L/min, about 87 L/min, about 87.25 L/min, about 87.5 L/min, about 87.75 L/min, about 88 L/min, about 88.25 L/min, about 88.5 L/min, about 88.75 L/min, about 89 L/min, about 89.25 L/min, about 89.5 L/min, about 89.75 L/min, about 90 L/min, about 90.25 L/min, about 90.5 L/min, about 90.75 L/min, about 91 L/min, about 91.25 L/min, about 91.5 L/min, about 91.75 L/min, about 92 L/min, about 92.25 L/min, about 92.5 L/min, about 92.75 L/min, about 93 L/min, about 93.25 L/min, about 93.5 L/min, about 93.75 L/min, about 94 L/min, about 94.25 L/min, about 94.5 L/min, about 94.75 L/min, about 95 L/min, about 95.25 L/min, about 95.5 L/min, about 95.75 L/min, about 96 L/min, about 96.25 L/min, about 96.5 L/min, about 96.75 L/min, about 97 L/min, about 97.25 L/min, about 97.5 L/min, about 97.75 L/min, about 98 L/min, about 98.25 L/min, about 98.5 L/min, about 98.75 L/min, about 99 L/min, about 99.25 L/min, about 99.5 L/min, about 99.75 L/min, or about 100 L/min, including all ranges and subranges therebetween.

In some embodiments, the oxygen source provides gas at a flow rate of at least about 1 mL/min, at least about 2 mL/min, at least about 3 mL/min, at least about 4 mL/min, at least about 5 mL/min, at least about 6 mL/min, at least about 7 mL/min, at least about 8 mL/min, at least about 9 mL/min, at least about 10 mL/min, at least about 11 mL/min, at least about 12 mL/min, at least about 13 mL/min, at least about 14 mL/min, at least about 15 mL/min, at least about 16 mL/min, at least about 17 mL/min, at least about 18 mL/min, at least about 19 mL/min, at least about 20 mL/min, at least about 21 mL/min, at least about 22 mL/min, at least about 23 mL/min, at least about 24 mL/min, at least about 25 mL/min, at least about 26 mL/min, at least about 27 mL/min, at least about 28 mL/min, at least about 29 mL/min, at least about 30 mL/min, at least about 31 mL/min, at least about 32 mL/min, at least about 33 mL/min, at least about 34 mL/min, at least about 35 mL/min, at least about 36 mL/min, at least about 37 mL/min, at least about 38 mL/min, at least about 39 mL/min, at least about 40 mL/min, at least about 41 mL/min, at least about 42 mL/min, at least about 43 mL/min, at least about 44 mL/min, at least about 45 mL/min, at least about 46 mL/min, at least about 47 mL/min, at least about 48 mL/min, at least about 49 mL/min, at least about 50 mL/min, at least about 51 mL/min, at least about 52 mL/min, at least about 53 mL/min, at least about 54 mL/min, at least about 55 mL/min, at least about 56 mL/min, at least about 57 mL/min, at least about 58 mL/min, at least about 59 mL/min, at least about 60 mL/min, at least about 61 mL/min, at least about 62 mL/min, at least about 63 mL/min, at least about 64 mL/min, at least about 65 mL/min, at least about 66 mL/min, at least about 67 mL/min, at least about 68 mL/min, at least about 69 mL/min, at least about 70 mL/min, at least about 71 mL/min, at least about 72 mL/min, at least about 73 mL/min, at least about 74 mL/min, at least about 75 mL/min, at least about 76 mL/min, at least about 77 mL/min, at least about 78 mL/min, at least about 79 mL/min, at least about 80 mL/min, at least about 81 mL/min, at least about 82 mL/min, at least about 83 mL/min, at least about 84 mL/min, at least about 85 mL/min, at least about 86 mL/min, at least about 87 mL/min, at least about 88 mL/min, at least about 89 mL/min, at least about 90 mL/min, at least about 91 mL/min, at least about 92 mL/min, at least about 93 mL/min, at least about 94 mL/min, at least about 95 mL/min, at least about 96 mL/min, at least about 97 mL/min, at least about 98 mL/min, at least about 99 mL/min, at least about 100 mL/min, at least about 105 mL/min, at least about 110 mL/min, at least about 115 mL/min, at least about 120 mL/min, at least about 125 mL/min, at least about 130 mL/min, at least about 135 mL/min, at least about 140 mL/min, at least about 145 mL/min, at least about 150 mL/min, at least about 155 mL/min, at least about 160 mL/min, at least about 165 mL/min, at least about 170 mL/min, at least about 175 mL/min, at least about 180 mL/min, at least about 185 mL/min, at least about 190 mL/min, at least about 195 mL/min, at least about 200 mL/min, at least about 225 mL/min, at least about 250 mL/min, at least about 275 mL/min, at least about 300 mL/min, at least about 325 mL/min, at least about 350 mL/min, at least about 375 mL/min, at least about 400 mL/min, at least about 425 mL/min, at least about 450 mL/min, at least about 475 mL/min, at least about 500 mL/min, at least about 525 mL/min, at least about 550 mL/min, at least about 575 mL/min, at least about 600 mL/min, at least about 625 mL/min, at least about 650 mL/min, at least about 675 mL/min, at least about 700 mL/min, at least about 725 mL/min, at least about 750 mL/min, at least about 775 mL/min, at least about 800 mL/min, at least about 825 mL/min, at least about 850 mL/min, at least about 875 mL/min, at least about 900 mL/min, at least about 925 mL/min, at least about 950 mL/min, at least about 975 mL/min, at least about 1 L/min, at least about 1.25 L/min, at least about 1.5 L/min, at least about 1.75 L/min, at least about 2 L/min, at least about 2.25 L/min, at least about 2.5 L/min, at least about 2.75 L/min, at least about 3 L/min, at least about 3.25 L/min, at least about 3.5 L/min, at least about 3.75 L/min, at least about 4 L/min, at least about 4.25 L/min, at least about 4.5 L/min, at least about 4.75 L/min, at least about 5 L/min, at least about 5.25 L/min, at least about 5.5 L/min, at least about 5.75 L/min, at least about 6 L/min, at least about 6.25 L/min, at least about 6.5 L/min, at least about 6.75 L/min, at least about 7 L/min, at least about 7.25 L/min, at least about 7.5 L/min, at least about 7.75 L/min, at least about 8 L/min, at least about 8.25 L/min, at least about 8.5 L/min, at least about 8.75 L/min, at least about 9 L/min, at least about 9.25 L/min, at least about 9.5 L/min, at least about 9.75 L/min, at least about 10 L/min, at least about 10.25 L/min, at least about 10.5 L/min, at least about 10.75 L/min, at least about 11 L/min, at least about 11.25 L/min, at least about 11.5 L/min, at least about 11.75 L/min, at least about 12 L/min, at least about 12.25 L/min, at least about 12.5 L/min, at least about 12.75 L/min, at least about 13 L/min, at least about 13.25 L/min, at least about 13.5 L/min, at least about 13.75 L/min, at least about 14 L/min, at least about 14.25 L/min, at least about 14.5 L/min, at least about 14.75 L/min, at least about 15 L/min, at least about 15.25 L/min, at least about 15.5 L/min, at least about 15.75 L/min, at least about 16 L/min, at least about 16.25 L/min, at least about 16.5 L/min, at least about 16.75 L/min, at least about 17 L/min, at least about 17.25 L/min, at least about 17.5 L/min, at least about 17.75 L/min, at least about 18 L/min, at least about 18.25 L/min, at least about 18.5 L/min, at least about 18.75 L/min, at least about 19 L/min, at least about 19.25 L/min, at least about 19.5 L/min, at least about 19.75 L/min, at least about 20 L/min, at least about 20.25 L/min, at least about 20.5 L/min, at least about 20.75 L/min, at least about 21 L/min, at least about 21.25 L/min, at least about 21.5 L/min, at least about 21.75 L/min, at least about 22 L/min, at least about 22.25 L/min, at least about 22.5 L/min, at least about 22.75 L/min, at least about 23 L/min, at least about 23.25 L/min, at least about 23.5 L/min, at least about 23.75 L/min, at least about 24 L/min, at least about 24.25 L/min, at least about 24.5 L/min, at least about 24.75 L/min, at least about 25 L/min, at least about 25.25 L/min, at least about 25.5 L/min, at least about 25.75 L/min, at least about 26 L/min, at least about 26.25 L/min, at least about 26.5 L/min, at least about 26.75 L/min, at least about 27 L/min, at least about 27.25 L/min, at least about 27.5 L/min, at least about 27.75 L/min, at least about 28 L/min, at least about 28.25 L/min, at least about 28.5 L/min, at least about 28.75 L/min, at least about 29 L/min, at least about 29.25 L/min, at least about 29.5 L/min, at least about 29.75 L/min, at least about 30 L/min, at least about 30.25 L/min, at least about 30.5 L/min, at least about 30.75 L/min, at least about 31 L/min, at least about 31.25 L/min, at least about 31.5 L/min, at least about 31.75 L/min, at least about 32 L/min, at least about 32.25 L/min, at least about 32.5 L/min, at least about 32.75 L/min, at least about 33 L/min, at least about 33.25 L/min, at least about 33.5 L/min, at least about 33.75 L/min, at least about 34 L/min, at least about 34.25 L/min, at least about 34.5 L/min, at least about 34.75 L/min, at least about 35 L/min, at least about 35.25 L/min, at least about 35.5 L/min, at least about 35.75 L/min, at least about 36 L/min, at least about 36.25 L/min, at least about 36.5 L/min, at least about 36.75 L/min, at least about 37 L/min, at least about 37.25 L/min, at least about 37.5 L/min, at least about 37.75 L/min, at least about 38 L/min, at least about 38.25 L/min, at least about 38.5 L/min, at least about 38.75 L/min, at least about 39 L/min, at least about 39.25 L/min, at least about 39.5 L/min, at least about 39.75 L/min, at least about 40 L/min, at least about 40.25 L/min, at least about 40.5 L/min, at least about 40.75 L/min, at least about 41 L/min, at least about 41.25 L/min, at least about 41.5 L/min, at least about 41.75 L/min, at least about 42 L/min, at least about 42.25 L/min, at least about 42.5 L/min, at least about 42.75 L/min, at least about 43 L/min, at least about 43.25 L/min, at least about 43.5 L/min, at least about 43.75 L/min, at least about 44 L/min, at least about 44.25 L/min, at least about 44.5 L/min, at least about 44.75 L/min, at least about 45 L/min, at least about 45.25 L/min, at least about 45.5 L/min, at least about 45.75 L/min, at least about 46 L/min, at least about 46.25 L/min, at least about 46.5 L/min, at least about 46.75 L/min, at least about 47 L/min, at least about 47.25 L/min, at least about 47.5 L/min, at least about 47.75 L/min, at least about 48 L/min, at least about 48.25 L/min, at least about 48.5 L/min, at least about 48.75 L/min, at least about 49 L/min, at least about 49.25 L/min, at least about 49.5 L/min, at least about 49.75 L/min, at least about 50 L/min, at least about 50.25 L/min, at least about 50.5 L/min, at least about 50.75 L/min, at least about 51 L/min, at least about 51.25 L/min, at least about 51.5 L/min, at least about 51.75 L/min, at least about 52 L/min, at least about 52.25 L/min, at least about 52.5 L/min, at least about 52.75 L/min, at least about 53 L/min, at least about 53.25 L/min, at least about 53.5 L/min, at least about 53.75 L/min, at least about 54 L/min, at least about 54.25 L/min, at least about 54.5 L/min, at least about 54.75 L/min, at least about 55 L/min, at least about 55.25 L/min, at least about 55.5 L/min, at least about 55.75 L/min, at least about 56 L/min, at least about 56.25 L/min, at least about 56.5 L/min, at least about 56.75 L/min, at least about 57 L/min, at least about 57.25 L/min, at least about 57.5 L/min, at least about 57.75 L/min, at least about 58 L/min, at least about 58.25 L/min, at least about 58.5 L/min, at least about 58.75 L/min, at least about 59 L/min, at least about 59.25 L/min, at least about 59.5 L/min, at least about 59.75 L/min, at least about 60 L/min, at least about 60.25 L/min, at least about 60.5 L/min, at least about 60.75 L/min, at least about 61 L/min, at least about 61.25 L/min, at least about 61.5 L/min, at least about 61.75 L/min, at least about 62 L/min, at least about 62.25 L/min, at least about 62.5 L/min, at least about 62.75 L/min, at least about 63 L/min, at least about 63.25 L/min, at least about 63.5 L/min, at least about 63.75 L/min, at least about 64 L/min, at least about 64.25 L/min, at least about 64.5 L/min, at least about 64.75 L/min, at least about 65 L/min, at least about 65.25 L/min, at least about 65.5 L/min, at least about 65.75 L/min, at least about 66 L/min, at least about 66.25 L/min, at least about 66.5 L/min, at least about 66.75 L/min, at least about 67 L/min, at least about 67.25 L/min, at least about 67.5 L/min, at least about 67.75 L/min, at least about 68 L/min, at least about 68.25 L/min, at least about 68.5 L/min, at least about 68.75 L/min, at least about 69 L/min, at least about 69.25 L/min, at least about 69.5 L/min, at least about 69.75 L/min, at least about 70 L/min, at least about 70.25 L/min, at least about 70.5 L/min, at least about 70.75 L/min, at least about 71 L/min, at least about 71.25 L/min, at least about 71.5 L/min, at least about 71.75 L/min, at least about 72 L/min, at least about 72.25 L/min, at least about 72.5 L/min, at least about 72.75 L/min, at least about 73 L/min, at least about 73.25 L/min, at least about 73.5 L/min, at least about 73.75 L/min, at least about 74 L/min, at least about 74.25 L/min, at least about 74.5 L/min, at least about 74.75 L/min, at least about 75 L/min, at least about 75.25 L/min, at least about 75.5 L/min, at least about 75.75 L/min, at least about 76 L/min, at least about 76.25 L/min, at least about 76.5 L/min, at least about 76.75 L/min, at least about 77 L/min, at least about 77.25 L/min, at least about 77.5 L/min, at least about 77.75 L/min, at least about 78 L/min, at least about 78.25 L/min, at least about 78.5 L/min, at least about 78.75 L/min, at least about 79 L/min, at least about 79.25 L/min, at least about 79.5 L/min, at least about 79.75 L/min, at least about 80 L/min, at least about 80.25 L/min, at least about 80.5 L/min, at least about 80.75 L/min, at least about 81 L/min, at least about 81.25 L/min, at least about 81.5 L/min, at least about 81.75 L/min, at least about 82 L/min, at least about 82.25 L/min, at least about 82.5 L/min, at least about 82.75 L/min, at least about 83 L/min, at least about 83.25 L/min, at least about 83.5 L/min, at least about 83.75 L/min, at least about 84 L/min, at least about 84.25 L/min, at least about 84.5 L/min, at least about 84.75 L/min, at least about 85 L/min, at least about 85.25 L/min, at least about 85.5 L/min, at least about 85.75 L/min, at least about 86 L/min, at least about 86.25 L/min, at least about 86.5 L/min, at least about 86.75 L/min, at least about 87 L/min, at least about 87.25 L/min, at least about 87.5 L/min, at least about 87.75 L/min, at least about 88 L/min, at least about 88.25 L/min, at least about 88.5 L/min, at least about 88.75 L/min, at least about 89 L/min, at least about 89.25 L/min, at least about 89.5 L/min, at least about 89.75 L/min, at least about 90 L/min, at least about 90.25 L/min, at least about 90.5 L/min, at least about 90.75 L/min, at least about 91 L/min, at least about 91.25 L/min, at least about 91.5 L/min, at least about 91.75 L/min, at least about 92 L/min, at least about 92.25 L/min, at least about 92.5 L/min, at least about 92.75 L/min, at least about 93 L/min, at least about 93.25 L/min, at least about 93.5 L/min, at least about 93.75 L/min, at least about 94 L/min, at least about 94.25 L/min, at least about 94.5 L/min, at least about 94.75 L/min, at least about 95 L/min, at least about 95.25 L/min, at least about 95.5 L/min, at least about 95.75 L/min, at least about 96 L/min, at least about 96.25 L/min, at least about 96.5 L/min, at least about 96.75 L/min, at least about 97 L/min, at least about 97.25 L/min, at least about 97.5 L/min, at least about 97.75 L/min, at least about 98 L/min, at least about 98.25 L/min, at least about 98.5 L/min, at least about 98.75 L/min, at least about 99 L/min, at least about 99.25 L/min, at least about 99.5 L/min, at least about 99.75 L/min, or at least about 100 L/min.

In some embodiments, the oxygen source comprises an aeration stone. In some embodiments, the aeration stone is located on the opening through which oxygen is released. In some embodiments, the aeration stone is a 0.5 micron stone.

In some embodiments, the container contains an opening that permits carbon dioxide (CO₂) to escape the liquid. In some embodiments, the container contains an opening that permits carbon monoxide (CO) to escape the liquid. In some embodiments the diameter of the opening is between about 1 μm and about 100 cm. In some embodiments the opening is a passive or actively controlled valve, latch or door.

In some embodiments, the apparatus comprises a container with inside capable of holding liquid. The present invention is compatible with any container. In some embodiments, containers that do not absorb, react, or otherwise alter the Cannabis composition are preferred. As used herein, a container is capable of holding liquid. In some embodiments, the container is a beaker, flask, or round bottom flask. In some embodiments, the container is glass. In some embodiments, the container comprises a volume of between about 1 milliliter (mL) and about 50 liters (L). For example, the container comprises about 1 mL, about 5 mL, about 10 mL, about 50 mL, about 100 mL, about 150 mL, about 200 mL, about 250 mL, about 300 mL, about 350 mL, about 400 mL, about 450 mL, about 500 mL, about 550 mL, about 600 mL, about 650 mL, about 700 mL, about 750 mL, about 800 mL, about 900 mL, about 1000 mL, about 1100 mL, about 1200 mL, about 1300 mL, about 1400 mL, about 1500 mL, about 1600 mL, about 1700 mL, about 1800 mL, about 1900 mL, about 2 L, about 2.5 L, about 3 L, about 4 L, about 5 L, about 6 L, about 7 L, about 8 L, about 9 L, about 10 L, about 11 L, about 12 L, about 13 L, about 14 L, about 15 L, about 16 L, about 17 L, about 18 L, about 19 L, about 20 L, about 21 L, about 22 L, about 23 L, about 24 L, about 25 L, about 26 L, about 27 L, about 28 L, about 29 L, about 30 L, about 31 L, about 32 L, about 33 L, about 34 L, about 35 L, about 36 L, about 37 L, about 38 L, about 39 L, about 40 L, about 41 L, about 42 L, about 43 L, about 44 L, about 45 L, about 46 L, about 47 L, about 48 L, about 49 L, or about 50 L, including all ranges and subranges therebetween. Industrial-scale apparatus may comprise larger containers, as necessary to house the THC source.

In some embodiments, the container comprises a Cannabis composition. In some embodiments, the Cannabis composition comprises a cannabinoid selected from the group consisting of THC, THCA, CBD, CBDA, and CBN (including combinations thereof). In some embodiments, the Cannabis composition comprises a terpene.

In some embodiments, the apparatus comprises a heat source. In some embodiments, the apparatus of the present disclosure is compatible with any heat source. In some embodiments, the heat source is a hot plate. In some embodiments, the heat source is a microwave emitter. I some embodiments, the heat source is a heat pump. In some embodiments, the heat source is a flame.

In some embodiments, the apparatus comprises a temperature monitor located so as to measure the temperature of the contents inside the container. In some embodiments, a temperature monitor is operably connected to the heat source. In some embodiments, the temperature monitor is configured to regulate the heat source so as to maintain the contents inside of the container at a temperature between about 130° C. and about 180° C. For example, the temperature monitor is configured to regulate the heat source so as to maintain the contents inside the container at a temperature of about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., about 148° C., about 149° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., or about 180° C., including all ranges and subranges therebetween. In some embodiments, the temperature monitor is configured to regulate the heat source so as to maintain the contents inside the container at a temperature of at least about 130° C., at least about 131° C., at least about 132° C., at least about 133° C., at least about 134° C., at least about 135° C., at least about 136° C., at least about 137° C., at least about 138° C., at least about 139° C., at least about 140° C., at least about 141° C., at least about 142° C., at least about 143° C., at least about 144° C., at least about 145° C., at least about 146° C., at least about 147° C., at least about 148° C., at least about 149° C., at least about 150° C., at least about 151° C., at least about 152° C., at least about 153° C., at least about 154° C., at least about 155° C., at least about 156° C., at least about 157° C., at least about 158° C., at least about 159° C., at least about 160° C., at least about 161° C., at least about 162° C., at least about 163° C., at least about 164° C., at least about 165° C., at least about 166° C., at least about 167° C., at least about 168° C., at least about 169° C., at least about 170° C., at least about 171° C., at least about 172° C., at least about 173° C., at least about 174° C., at least about 175° C., at least about 176° C., at least about 177° C., at least about 178° C., at least about 179° C., or at least about 180° C.

In some embodiments, the apparatus comprises a stirring mechanism. In some embodiments, the stirring mechanism is capable of stirring contents placed inside the container. In some embodiments, the stirring mechanism can be any known means for stirring. Persons with skill in the art will be familiar with ways to stir solutions. In some embodiments, the stirring mechanism comprises a mechanized paddle, blade, or arm. In some embodiments, the stirring mechanism stirs matter within the container by moving the container itself (e.g., by shaking or rotating). In some embodiments, the stirring mechanism is a magnetic mechanism capable of rotating a magnetic stir bar. In some embodiments, the stirring mechanism creates a vortex motion, which spreads oil to the surface of the container for even heat and oxygen displacement within the surface of the reaction vessel.

In some embodiments, the stirring mechanism spins at a speed between about 100 rpm and about 1200 rpm. For example, the stirring mechanism spins at about 100 rpm, about 150 rpm, about 200 rpm, about 250 rpm, 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, about 1000 rpm, about 1050 rpm, about 1100 rpm, about 1150 rpm, or about 1200 rpm, including all values and subranges therebetween. In some embodiments, the stirring mechanism spins at at least about 100 rpm, at least about 150 rpm, at least about 200 rpm, at least about 250 rpm, 300 rpm, at least about 350 rpm, at least about 400 rpm, at least about 450 rpm, at least about 500 rpm, at least about 550 rpm, at least about 600 rpm, at least about 650 rpm, at least about 700 rpm, at least about 750 rpm, at least about 800 rpm, at least about 850 rpm, at least about 900 rpm, at least about 950 rpm, at least about 1000 rpm, at least about 1050 rpm, at least about 1100 rpm, at least about 1150 rpm, or at least about 1200 rpm, including all values and subranges therebetween. In some embodiments, the speed of the vortex is between about 300 rpm and about 900 rpm. For example, the speed of the vortex is about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, including all values and subranges therebetween. In some embodiments, the speed of the vortex is between about 550 rpm and 700 rpm, for example, about 550 rpm, about 560 rpm, about 570 rpm, about 580 rpm, about 590 rpm, about 600 rpm, about 610 rpm, about 620 rpm, about 630 rpm, about 640 rpm, about 650 rpm, about 660 rpm, about 670 rpm, about 680 rpm, about 690 rpm, or about 700 rpm, including all values and subranges therebetween. In some embodiments, the speed of the vortex is between about 400 rpm and about 800 rpm. For example, the speed of the vortex is about 400 rpm, about 410 rpm, about 420 rpm, about 430 rpm, about 440 rpm, about 450 rpm, about 460 rpm, about 470 rpm, about 480 rpm, about 490 rpm, about 500 rpm, about 510 rpm, about 520 rpm, about 530 rpm, about 540 rpm, about 550 rpm, about 560 rpm, about 570 rpm, about 580 rpm, about 590 rpm, about 600 rpm, about 610 rpm, about 620 rpm, about 630 rpm, about 640 rpm, about 650 rpm, about 660 rpm, about 670 rpm, about 680 rpm, about 690 rpm, about 700 rpm, about 710 rpm, about 720 rpm, about 730 rpm, about 740 rpm, about 750 rpm, about 760 rpm, about 770 rpm, about 780 rpm, about 790 rpm, or about 800 rpm, including all values and subranges therebetween.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1: An Apparatus for Conversion of a THC Source to CBN

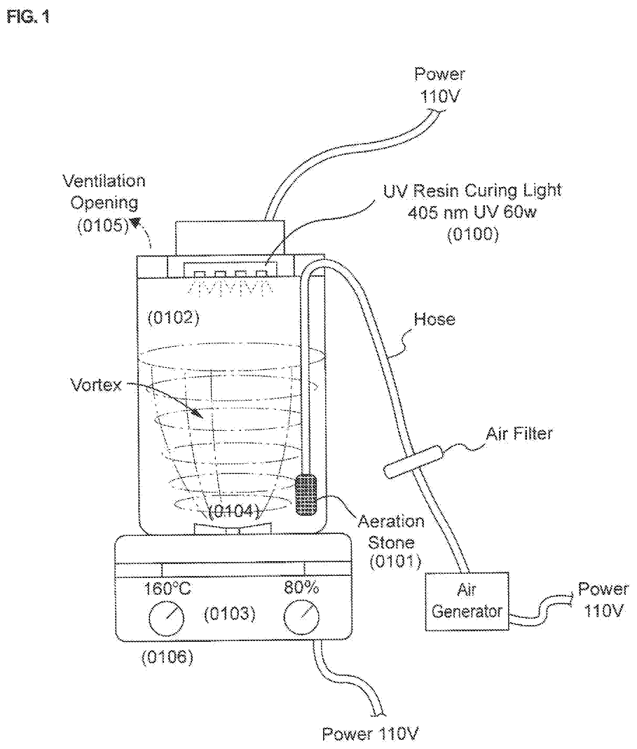

An apparatus for converting a THC source to CBN was developed (FIG. 1). The apparatus contained the following:

(a) an ultraviolet (UV) light source (0100);

(b) an oxygen source with an opening through which oxygen is released (0101);

(c) a container with an inside capable of holding liquid (0102);

(d) a heat source (0103);

(e) a stirring mechanism (0104);

(f) an opening for CO₂ release (0105); and,

(g) a temperature monitor (0106).

The UV light source enhances the conversion of a THC source to CBN. The oxygen source provides oxygen to the inside of the container. A purpose of the oxygen source is to aromatize the THC to CBN. The UV light is a resin-curing light with peak emissions at about 405 nm LED source, which is mounted on top of the container, pointing into the container. The container holds liquid, for example, a Cannabis composition. The heat source heats up the container containing a liquid. By heating up the container, the heat source enhances reactions within the container, for example, the conversion of THC to CBN, the conversion of THCA to THC, or the conversion of CBDA to CBD. The apparatus also contains a stirring mechanism that is capable of stirring liquids placed in the container. A temperature monitor enables monitoring of the temperature of the liquid in the container.

### Example 2: Conversion of THCA to CBN

Solid THCA isolate (about 1300 grams of THCA) is the starting material used for the conversion of THCA to CBN (i.e. the THC source).

The apparatus of Example 1 is used to convert THCA to CBN. The THCA isolate described above is placed into a glass flask, e.g. the container of the apparatus described in Example 1. The container is heated for 45 minutes to 60 minutes at 130° C. until cannabinoid crystals are fully dissolved. During this process, the THCA will also begin to decarboxylate to THC. The heat is lowered to 110° C. until air bubbles in the liquid dissipate. The heat is increased to between about 160° C. and 170° C., and the glass flask containing the (now) decarboxylated liquid THC (e.g. the THC source or Cannabis composition) is stirred using a magnetic stir bar. The rate of stirring is gradually increased from 100 rpm to 900 rpm to create a continuous vortex of Cannabis composition.

The container containing the Cannabis composition is exposed to oxygen via an oxygen diffuser placed about 1 inch to 2 inches below the surface of the now liquid THC source. The enhanced oxygen system is turned on to increase oxygen injection from 10% to 60%. The container containing the Cannabis composition is exposed to between 60 W and 400 W of 405 nm UV light. The UV light is directed into the continuous vortex.

The apparatus runs until the conversion of THC to CBN is complete. The conversion rate of THC to CBN ranges from between 10 mg of THC source /hour to about 50 mg of THC source/minute. The conversion takes between 10 to 130 hours to complete.

The resultant CBN oil reaches a CBN titer of about 30% to 60% purity, depending on the amount of THC reactant used. The CBN oil is refined to a purity of between 50% w/w and 90% w/w CBN using Wipe-Film (Short Path) Distillation or Evaporation. The CBN oil is refined to a purity of between 90% w/w and 99% w/w CBN using ultra-high preperformance supercritical fluid chromatography/photodiode array-mass spectrometry (UHPSFC/PDA-MS).

### Example 3: Conversion of Cannabis Composition Comprising THC, THCA, CBCA, CBG, and CBGA to CBN

The methods of Example 2 are utilized to convert a composition containing THC, THCA, CBCA, CBG, and CBGA to CBN.

The contents of the Cannabis composition are shown in Table 1.

TABLE 1

| Cannabinoid | mg/g | % (w/w) |
|---|---|---|
| Δ9THC | 5.729 | 0.5729 |
| Δ8THC | ND | ND |
| THCa | 943.812 | 94.3812 |
| THCV | ND | ND |
| THCVa | 3.773 | 0.3773 |
| CBD | ND | ND |
| CBDa | ND | ND |
| CBDV | ND | ND |
| CBDVa | ND | ND |
| CBG | 0.572 | 0.0572 |
| CBGa | 10.963 | 1.0963 |
| CBL | ND | ND |
| CBN | ND | ND |
| CBC | ND | ND |
| CBCa | 3.669 | 0.3669 |
| Sum of Cannabinoids: | 968.518 | 96.8518 |
| Total THC | 833.452 | 83.3452 |
| (Δ9THC + 0.877*THCa) | | |
| Total CBD | ND | ND |
| (CBD + 0.877*CBDa) | | |
| Δ9THC/unit | Action | |
| | Limit (mg): | |
| | 1000 | |

### Example 4: Conversion of Cannabis Composition Comprising THC, THCA, CBN, BCD, and CBG to CBN

The apparatus of Example 1 is used to convert a THC source to CBN. The THC source contained Δ-9-tetrahydrocannabinol (Δ9THC), THCA, CBN, BCD, and CBG to CBN (Table 2). This example was conducted to validate the method's conversion of THC to CBN from complex THC sources, comprising multiple cannabinoids. This example also tracked the conversion of THC to CBN over time, to provide kinetic information on the reaction.

The THC source is placed into a glass flask, e.g. the container of the apparatus described in Example 1. The container is heated for 45 minutes to 60 minutes at 130° C. to melt any cannabinoid crystals and to decarboxylate the THCA to THC. The heat is increased to between about 162° C. and 165° C., and the glass flask containing the (now) decarboxylated liquid THC (e.g. the Cannabis composition) is stirred using a magnetic stir bar. The rate of stirring is gradually increased from 400 rpm to 800 rpm to create a continuous vortex of Cannabis composition.

The container containing the Cannabis composition is exposed to oxygen via an oxygen diffuser placed within 5 inches of the top of the liquid. The enhanced oxygen system is turned on to increase oxygen injection from 10% to 60%. The container containing the Cannabis composition is exposed to between 60 W and 400 W of 405 nm UV light. The UV light is directed into the continuous vortex.

The amount of each of the aforementioned cannabinoids over time is shown in Table 2. All of the Δ9THC was converted to CBN within 46.17 hours. The yield of CBN was 0.502 g CBN per g of THC. The percent yield of CBN obtained from Δ9THC was 50.9%.

TABLE 2

| Time | Cannabinoid (% w/w) | | | | |
|---|---|---|---|---|---|
| (hours) | CBG | CBD | CBN | Δ9THC | Δ8THC |
| 0 | 2.98 | 0.36 | 0.44 | 90.54 | 0 |
| 1.9 | 2.807 | 0.31 | 1.157 | 79.49 | 0 |
| 3.53 | 2.851 | 0.336 | 2.483 | 75.42 | 0 |
| 21.45 | 2.516 | 0.278 | 25.94 | 28.96 | 0.9 |
| 24.02 | 2.63 | 0.42 | 30.38 | 23.81 | 1.12 |
| 28.58 | 2.51 | 0.4 | 35.2 | 15.48 | 1.19 |
| 46.17 | 1.71 | 0.31 | 45.52 | 0 | 1.19 |

### Example 5: Effect of THC Source on Conversion to CBN

The apparatus of Example 1 was used to convert various THC sources to CBN. The THC sources that were evaluated included: (i) a THC-A isolate, (ii) a THC distillate, (iii) a full spectrum rosin, and (iv) a fully decarb rosin.

Table 3 shows characteristics of the starting compositions, parameters that were applied to the apparatus, and the results. Each input source was fully converted to CBN within 40 to about 60 hours. The amount of CBN obtained after conversion (total amount of CBN) and after a subsequent purification via fractional distillation (Final CBN %) is shown in Table 3.

TABLE 3

| | | | Apparatus | | | | Results | | | | | | |
|---|---|---|---|---|---|---|---|---|---|---|---|---|---|
| | | | | | UV | | | | | | | Total | |
| | Starting Material | | | | Light | | | | | | | % of | |
| | | Starting | | | (315 | | | | | | | Volume | |
| | | Amount | | | nm- | Increased | | | | Total | | Lost | |
| | Starting | of | | Stir | 415 | Oxygen | | Final | Total | % | Final | From | |
| Input | Percentage | Material | Temperature | Speed | nm) | (Percentage | Time | % of | % of | volume | CBN | Starting | % |
| Source | of THC | (grams) | (Celsius) | (rpm) | (Watts) | oxygen) | (hrs) | THC | CBN | lost | % | Input | yield |
| THC-A | 95-99 | 500 | 160-170 | 550- | 30 | 10-30 | 40- | 0% | 50-55 | Under | 80- | Under 25 | 51- |
| Isolate | | | | 700 | | | 50 | | | 10 | 85% | | 59 |
| THC | 85-90 | 500 | 160-170 | 550- | 30 | 10-30 | 45- | Under | 40-45 | Under | 80- | Under 30 | 45- |
| Distillate | | | | 700 | | | 50 | 1% | | 10 | 85% | | 53 |
| Full | 75-85 | 500 | 165-175 | 550- | 30 | 10-30 | 45- | Under | 35-45 | Under | 70- | Under 35 | 41- |
| spectrum | | | | 700 | | | 55 | 2% | | 15 | 80% | | 61 |
| rosin | | | | | | | | | | | | | |
| Fully | 55-60 | 500 | 165-180 | 550- | 30 | 10-30 | 50- | Under | 20-30 | Under | 70- | Under 40 | 34- |
| decarb | | | | 700 | | | 60 | 3% | | 20 | 80% | | 55 |
| rosin | | | | | | | | | | | | | |

### Example 6: Effect of Amount of Starting Material on Conversion of THC to CBN

The apparatus of Example 1 is used to convert various amounts of a THC source to CBN. The container of the apparatus has a volume of 2 L. The following amounts of THC source are evaluated: (i) 500-750 g of THC source; (ii) less than 500 g of THC source; (iii) >750 g of THC source; and (iv) >1 kg of THC source.

The various amounts of THC source are converted to CBN using the method described in Example 2. The percent yield of CBN from the THC source is evaluated.

### Example 7: Effect of THC Source, UV Light, Heat, Oxygen, and Stirring on Conversion of a THC Source to CBN

A THC source is provided. Various THC sources are evaluated (e.g., CBD, CBDA, THC, and THCA). The effect of (i) exposure to UV light (wavelength of 315-415 nm), (ii) exposure to heat (130° C.-180° C.), (iii) exposure to oxygen (10-30%), and (iv) stirring (speed of 550 rpm-700 rpm) on conversion of the THC source to CBN is evaluated. Briefly, the THC source is exposed to each of the aforementioned parameters, alone or in combination, and the yield or percent yield of CBN is evaluated at various time points, such as, 1 hour, 2 hours, 3 hours, 4 hours, 5 hours, 6 hours, 12 hours, 18 hours, 24 hours, 30 hours, 36 hours, 42 hours, 46 hours, and 48 hours. The rate constant for production of CBN is also evaluated.

Table 4 shows various reactions evaluated. An “x” indicates that the reaction is exposed to a particular parameter.

TABLE 4

| | | UV | | |
|---|---|---|---|---|
| Reaction | Stirring | light | Oxygen | Heat |
| 1 | x | | | |
| 2 | | x | | |
| 3 | | | x | |
| 4 | | | | x |
| 5 | x | x | | |
| 6 | x | | x | |
| 7 | x | | | x |
| 8 | | x | x | |
| 9 | | x | | x |
| 10 | | | x | x |
| 11 | x | x | x | |
| 12 | x | | x | x |
| 13 | | x | x | x |
| 14 | x | x | | x |

The effect of temperature on conversion of a THC source to CBN is also evaluated. The yield and percent yield of CBN is evaluated after exposure of a THC source to the following temperatures: 130° C., 135° C., 140° C., 145° C., 150° C., 155° C., 160° C., 165° C., 170° C., 175° C., or 180° C.

The effect of the wavelength of UV light on conversion of a THC source to CBN is also evaluated. The yield and percent yield of CBN is evaluated after exposure of a THC source to the following wavelengths: 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, and 415 nm, including ranges and subranges therein.

The effect of the power of the UV light on conversion of a THC source to CBN is also evaluated. The yield and percent yield of CBN is evaluated after exposure of a THC source to the following powers: 10 Watts, 15 Watts, 20 Watts, 25 Watts, 30 Watts, 35 Watts, 40 Watts, 45 Watts, 50 Watts, 55 Watts, and 60 Watts.

The effect of exposure to oxygen on conversion of a THC source to CBN is also evaluated. The yield and percent yield of CBN is evaluated after exposure of a THC source to the following amounts of oxygen: 1%, 5%, 10%, 15%, 20%, 25%, 30%, 35%, 40%, 45%, and 50%. The effect of oxygen flow rate (1 mL/min-100 L/min) on the conversion of a THC source to CBN is also evaluated. The amount of oxygen may refer to a percent increase as compared to the atmosphere or to an absolute amount of oxygen.

The effect of stirring on conversion of a THC source to CBN is also evaluated. The yield and percent yield of CBN is evaluated after the THC source is stirred at one of the following speeds: 350 rpm, 375 rpm, 400 rpm, 425 rpm, 450 rpm, 475 rpm, 500 rpm, 525 rpm, 550 rpm, 575 rpm, 600 rpm, 625 rpm, 650 rpm, 675 rpm, 700 rpm, 725 rpm, 750 rpm, 775 rpm, 800 rpm, 825 rpm, 850 rpm, 875 rpm, and 900 rpm.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Additional Embodiments of the Disclosure

The following embodiments are also envisioned by the present disclosure:
1. A method for converting a Δ⁹-tetrahydrocannabinol (THC) source to cannabinol (CBN), said method comprising the steps of:

a) providing a THC source, an ultra violet (UV) light source, and a heat source;

b) exposing the THC source to UV light from the UV light source and heating the THC source using the heat source for a time period sufficient to convert THC in the THC source to CBN.
1.1. The method of embodiment 1, wherein an oxygen flow source is provided in step (a), and wherein step (b) comprises exposing the THC source to exogenous oxygen from the oxygen source.
1.2. The method of embodiment 1, wherein the THC source is mixed/agitated/stirred during step (b).
1.3. A method for converting a Δ⁹-tetrahydrocannabinol (THC) source to cannabinol (CBN), said method comprising the steps of:

a) providing a THC source, an ultra violet (UV) light source, a heat source, and an oxygen source;

b) exposing the THC source to UV light from the UV light source, exogenous oxygen from the oxygen source, and heat from the heat source for a time period sufficient to convert THC in the THC source to CBN.
2. The method of any one of embodiments 1-1.3, wherein the UV light comprises long-wave UV light.
2.1. The method of any one of embodiments 1-1.3, wherein the UV light comprises UV-A light.
3. The method of any one of embodiments 1-2.1, wherein the UV light comprises light at a wavelength between about 315 nm and about 415 nm.
4. The method of any one of embodiments 1-2.1, wherein the UV light comprises light at a wavelength of about 403 nm.
4.1. The method of any one of embodiments 1-4, wherein the UV light has an irradiance in the UV-A range, of greater than 10 W/m².
5. The method of any one of embodiments 1-4.1, wherein step (b) is conducted at a temperature greater than 100° C., 110° C., 120° C., or 130° C.
5.1. The method of any one of embodiments 1-4.1, wherein step (b) is conducted at a temperature of between about 130° C. and about 180° C.
6. The method of any one of embodiments 1-4.1, wherein step (b) is conducted at a temperature of between about 150° C. and about 170° C.
7. The method of any one of embodiments 1-4.1, wherein step (b) is conducted at a temperature of about 160° C.
8. The method of any one of embodiments 1-7, wherein the THC source is stirred during step (b).
9. The method of any one of embodiments 1-8, wherein the time period is at least about 1, 2, 3, 4, 5, 6, 7, 8, 9, or 10 hours.
10. The method of any one of embodiments 1-8, wherein the time period is at least about 24, 48, or 72 hours.
11. The method of any one of embodiments 1-10, wherein the THC source comprises tetrahydrocannabinolic acid (THCA), which is then converted to THC in step (b).
11.1. The method of any one of embodiments 1-11, comprising isolating the CBN produced from the method.
11.2. The method of claim 11.1, wherein the isolating step comprises applying chemical separation techniques to recover CBN from the THC source after the time period sufficient to produce CBN.
12. The method of any one of embodiments 1-10, wherein the THC source comprises cannabidiol (CBD) and/or cannabidiolic acid (CBDA).
12.1. The method of any one of embodiments 1-12, wherein step (b) produces a CBN percent yield of at least 30%, 40%, or 50%.
12.2. The method of any one of embodiments 1-12.1, wherein step (b) produces a CBN cannabinoid titer of at least 30%, 40%, or 50%.
12.3. The method of any one of embodiments 1-12.2, wherein conversion of THC to CBN in step(b) exhibits a rate constant of at least about 0.06 hr⁻¹.
12.4. The method of any one of embodiments 1-12.3, wherein the exogenous oxygen from the oxygen source is delivered inside the THC source, so as to produce exogenous oxygen bubbles.
12.5. The method of any one of embodiments 1-12.4, wherein the exogenous oxygen comprises air with an oxygen content of at least 21% by weight.
12.6. The method of any one of embodiments 1-12.4, wherein the exogenous oxygen comprises air with an enriched oxygen content that is greater than the oxygen content of atmospheric air.
12.7. A method for converting a Δ⁹-tetrahydrocannabinol (THC) source to cannabinol (CBN), said method comprising the steps of:

a) heating a THC source, to at least 100° C., 110° C., 120° C., or 130° C.; and

b) shining ultraviolet (UV) light on the THC source;

wherein step (a) and (b) are conducted for a time period sufficient to convert THC in the THC source to CBN.
12.8. The method of embodiment 12.7, comprising step c) contacting the THC source with exogenous oxygen, wherein steps (a)-(c) are conducted for a time period sufficient to convert THC in the THC source to CBN.
12.9. The method of embodiment 12.8, wherein the exogenous oxygen is delivered inside the THC source, so as to produce exogenous oxygen bubbles within the THC source.
12.10. The method of any one of embodiments 12.7-12.9, wherein the THC source is mixed/agitated/stirred during the time period sufficient to convert THC in the THC source to CBN.
12.11. The method of any one of embodiments 12.7-12.10, wherein the UV light comprises long-wave UV light.
12.12. The method of any one of embodiments 12.7-12.10, wherein the UV light comprises UV-A light.
12.13. The method of any one of embodiments 12.7-12.10, wherein the UV light comprises light at a wavelength between about 315 nm and about 415 nm.
12.14. The method of any one of embodiments 12.7-12.10, wherein the UV light comprises light at a wavelength of about 403 nm.
12.15. The method of any one of embodiments 12.7-12.14, wherein the UV light has an irradiance in the UV-A range, of greater than 10 W/m².
12.16. The method of any one of embodiments 12.7- 12.15, wherein step (a) comprises heating the THC source to a temperature greater than 100° C., 110° C., 120° C., or 130° C.
12.17. The method of any one of embodiments 12.7- 12.16, wherein step (b) comprises heating the THC source to a temperature between about 130° C. and about 180° C.
12.18. The method of any one of embodiments 12.7- 12.17, wherein step (b) comprises heating the THC source to a temperature between about 150° C. and about 170° C.
12.19. The method of any one of embodiments 12.7- 12.18, wherein step (b) comprises heating the THC source to a temperature of about 160° C.
12.20. The method of any one of embodiments 12.7- 12.19, wherein the THC source is stirred during the time period sufficient to convert THC in the THC source to CBN.
12.21. The method of any one of embodiments 12.7- 12.20, wherein the time period is at least about 1, 2, 3, 4, 5, 6, 7, 8, 9, or 10 hours.
12.22. The method of any one of embodiments 12.7- 12.20, wherein the time period is at least about 24, 48, or 72 hours.
12.23. The method of any one of embodiments 12.7- 12.22, wherein the THC source comprises tetrahydrocannabinolic acid (THCA), which is then converted to THC in step (b).
12.24. The method of any one of embodiments 12.7- 12.23, wherein the THC source comprises cannabidiol (CBD) and/or cannabidiolic acid (CBDA).
12.25. The method of any one of embodiments 12.7- 12.24, wherein the method produces a CBN percent yield of at least 30%, 40%, or 50%.
12.26. The method of any one of embodiments 12.7- 12.25, wherein the method produces a CBN cannabinoid titer of at least 30%, 40%, or 50%.
12.27. The method of any one of embodiments, 12.7-12.26, wherein conversion of THC to CBN in step(b) exhibits a rate constant of at least about 0.06 hr⁻¹.
13. An apparatus for converting a Δ⁹-tetrahydrocannabinol (THC) source to cannabinol (CBN), said apparatus comprising:
   a) a container with an inside compartment capable of holding liquid;
   b) an ultra violet (UV) light source located so as to shine UV light to the inside compartment of the container;
   c) an exogenous oxygen source with an opening through which oxygen is released into the container
   d) a heat source located so as to be able to heat the container.
14. The apparatus of embodiment 13, wherein the apparatus comprises a stirring mechanism.
15. The apparatus of embodiment 14, wherein the stirring mechanism is capable of stirring materials placed inside the container.
16. The apparatus of any one of embodiments 13-14, wherein the stirring mechanism is a magnetic mechanism capable of rotating a magnetic stir bar.
16.1. The apparatus of embodiment 13, wherein the apparatus comprises a mechanism capable of agitating the container.
16.2. The apparatus of embodiment 13, wherein the apparatus comprises a pump capable of circulating contents in the container.
16.3. The apparatus of embodiment 13, wherein the apparatus comprises a motor or servo connected to the container so as to be able to rotate or invert the container.
16.4. The apparatus of embodiment 16.1, wherein the apparatus comprises a rotary shaker base capable of rotating the container.
17. The apparatus of any one of embodiments 13-16.3, wherein the container contains an opening or valve that permits CO₂ to escape the container.
18. The apparatus of any one of embodiments 13-17, wherein the UV light source produces long wave UV.
18.1 The apparatus of any one of embodiments 13-18, wherein the UV light source produces UV-A light.
19. The apparatus of any one of embodiments 13-18, wherein the UV light source produces light at a wavelength between 315 nm and 415 nm.
20. The apparatus of any one of embodiments 13-18, wherein the UV light source produces light at 403 nm.
20.1 The apparatus of any one of embodiments 13-20, wherein the UV light source produces an irradiance in the UV-A range, of greater than 10 W/m².
21. The apparatus of any one of embodiments 13-20.1, wherein the apparatus comprises a temperature monitor located so as to measure the temperature of contents inside the container.
22. The apparatus of embodiment 21, wherein the temperature monitor is operably connected to the heat source.
23. The apparatus of embodiments 21 or 22, wherein the temperature monitor is configured to regulate the heat source so as to maintain the contents inside of the container at a temperature between about 130° C. and about 180° C.
24. The method of any one of embodiments 13-23, wherein the exogenous oxygen source is configured to deliver oxygen inside the container.
25. The method of any one of embodiments 13-24, wherein the exogenous oxygen source is configured to deliver oxygen to contents inside the container, thereby generating exogenous oxygen bubbles in the contents.
26. The apparatus of any one of embodiments 13-25, wherein the exogenous oxygen source comprises an aeration stone.
27. The apparatus of embodiment 26, wherein the aeration stone is located on the opening through which oxygen is released.
28. The apparatus of embodiments 26 or 27, wherein the aeration stone is a 0.5 μm stone.
29. The apparatus of any one of embodiments 13-28, wherein the container comprises a cannabinoid selected from the group consisting of Δ⁹-tetrahydrocannabinol (THC), tetrahydrocannabinolic acid (THCA), cannabidiol (CBD), cannabidiolic acid (CBDA), and cannabinol (CBN).
30. The apparatus of any one of embodiments 13-29, wherein the container comprises THC source.
31. A system comprising:
   a) an electronic device, the electronic device including at least a memory and a processor operatively coupled to the memory and configured to execute instructions stored on the memory;
   b) a container with an inside compartment capable of holding liquid;
   c) an ultra violet (UV) light source in communication with the processor, said UV light located so as to shine UV light to the inside compartment of the container;
   d) an exogenous oxygen source in communication with the processor, said exogenous oxygen source with an opening through which oxygen is released into the container; and
   e) a heat source in communication with the processor, said heat source located so as to be able to heat the container;
      wherein the processor is configured to:
   i) expose the inside compartment of the container and any contents therein to UV light from the UV light source; and
   ii) heat the inside compartment of the container and any contents therein;
      wherein steps (i) and (ii) are carried out for a pre-determined period of time.
32. The system of embodiment 31, wherein the system further comprises f) an oxygen flow source; and wherein the processor is further configured to iii) expose the inside compartment of the container and any contents therein to exogenous oxygen from the oxygen source; wherein steps (i-iii) are carried out for a pre-determined period of time.
33. The system of any one of embodiment 31-32, wherein the system further comprises g) a means for mixing contents on the inside compartment of the container; and wherein the processor is further configured to iv) mix the contents; wherein steps (i-iv) are carried out for a pre-determined period of time.
34. The system of any one of embodiments 31-33, wherein the UV light comprises long-wave UV light.
35. The system of any one of embodiments 31-34, wherein the UV light comprises UV-A light.
36. The system of any one of embodiments 31-35, wherein the UV light comprises light at a wavelength between about 315 nm and about 415 nm.
37. The system of any one of embodiments 31-36, wherein the UV light comprises light at a wavelength of about 403 nm.
38. The system of any one of embodiments 31-37, wherein the UV light has an irradiance in the UV-A range, of greater than 10 W/m².
39. The system of any one of embodiments 31-38, wherein the heat source heats the inside of the container and any contents therein to a temperature greater than 100° C., 110° C., 120° C., or 130° C.
40. The system of any one of embodiments 31-38, wherein the heat source heats the inside of the container and any contents therein to a temperature of between about 130° C. and about 180° C.
41. The system of any one of embodiments 31-38, wherein the heat source heats the inside of the container and any contents therein to a temperature of between about 150° C. and about 170° C.
42. The system of any one of embodiments 31-38, wherein the heat source heats the inside of the container and any contents therein to a temperature of about 160° C.
43. The system of any one of embodiments 33-42, wherein the means for mixing contents inside the compartment of the container is a stirrer.
44. The system of any one of embodiments 31-43, wherein the pre-determined period of time is at least about 1, 2, 3, 4, 5, 6, 7, 8, 9, or 10 hours.
45. The system of any one of embodiments 31-43, wherein the pre-determined period of time is at least about 24, 48, or 72 hours.
46. The system of any one of embodiments 31-45, wherein the container comprises THC.
47. The system of any one of embodiments 31-46, wherein the system converts the THC to CBN.
48. The system of any one of embodiments 31-47, wherein the exogenous oxygen from the oxygen source is delivered inside contents inside the container, so as to produce exogenous oxygen bubbles.

What is claimed is:

1. A method for converting Δ⁹-tetrahydrocannabinol (THC) to cannabinol (CBN), said method comprising the steps of:
(a) providing THC, an ultra violet (UV) light source, and a heat source;
(b) exposing the THC to UV light from the UV light source and heating the THC using the heat source for a time period sufficient to convert THC to CBN.

2. The method of claim 1, wherein an oxygen flow source is provided, and wherein step (b) comprises exposing the THC to exogenous oxygen from the oxygen source.

3. The method of claim 2, wherein the exogenous oxygen from the oxygen source is delivered inside the THC source, so as to produce exogenous oxygen bubbles.

4. The method of claim 2, wherein the exogenous oxygen comprises air with an oxygen content of at least 21% by weight.

5. The method of claim 2, wherein the exogenous oxygen comprises air with an enriched oxygen content that is greater than the oxygen content of atmospheric air.

6. The method of claim 1, wherein the THC is stirred during step (b).

7. The method of claim 1, wherein the UV light comprises long-wave UV light.

8. The method of claim 1, wherein the UV light comprises UV-A light.

9. The method of claim 1, wherein the UV light comprises light at a wavelength between about 315 nm and about 415 nm.

10. The method of claim 9, wherein the UV light comprises light at a wavelength of about 405 nm.

11. The method of claim 1, wherein the UV light has an irradiance in the UV-A range, of greater than 10 W/m².

12. The method of claim 1, wherein the THC is heated to a temperature greater than 100° C.

13. The method of claim 1, wherein the THC is heated to a temperature of between about 130° C. and about 180° C.

14. The method of claim 1, wherein the THC is heated to a temperature of between about 160° C. and about 180° C.

15. The method of claim 14, wherein the THC is heated to a temperature of between about 162° C. and 165° C.

16. The method of claim 1, comprising isolating the CBN produced from the method.

17. The method of claim 16, wherein the isolating step comprises applying chemical separation techniques to recover CBN from the THC source after the time period sufficient to produce CBN.

18. The method of claim 1, wherein step (b) produces a CBN percent yield of at least 30%.

19. The method of claim 18, wherein step (b) produces a CBN percent yield of at least 40%.

20. The method of claim 1, wherein step (b) produces a CBN cannabinoid titer of at least 30%.

21. The method of claim 20, wherein step (b) produces a CBN cannabinoid titer of at least 50%.

22. The method of claim 20, wherein step (b) produces a CBN cannabinoid titer of at least 70%.

23. The method of claim 20, wherein step (b) produces a CBN cannabinoid titer of at least 90%.

24. The method of claim 1, wherein the THC is comprised within a composition and wherein after performing the method, the composition comprises less than 0.3% THC by weight.

25. A method for converting Δ⁹-tetrahydrocannabinol (THC) to cannabinol (CBN), said method comprising the steps of:
(a) providing THC, an ultra violet (UV) light source, a heat source, and an oxygen source;
(b) exposing the THC source to UV light from the UV light source, exogenous oxygen from the oxygen source, and heat from the heat source for a time period sufficient to convert THC to CBN;
wherein the THC is stirred in step (b).

26. A method for converting Δ⁹-tetrahydrocannabinol (THC) to cannabinol (CBN), said method comprising the steps of:
(a) providing THC, an ultra violet (UV) light source, a heat source, and an oxygen source;
(b) exposing the THC to UV light from the UV light source, exogenous oxygen from the oxygen source, and heat from the heat source for a time period sufficient to convert THC to CBN;
wherein the UV light comprises light at a wavelength between about 315 nm and about 415 nm; wherein the time period is at least 5 hours.

27. The method of claim 6, wherein the THC is stirred at a speed between about 400 and about 800 rpm.

28. The method of claim 25, wherein the THC is stirred at a speed between about 400 and about 800 rpm.

29. The method of claim 1, wherein the THC is provided in the form of Δ⁹-tetrahydrocannabinolic acid (THCA), which is converted to THC during step (b) of the method.

30. The method of claim 25, wherein the THC is provided in the form of Δ⁹-tetrahydrocannabinolic acid (THCA), which is converted to THC during step (b) of the method.
